SEQUENCE LISTING (14 sequences)

SEQ ID NO: 1 - DNA, 21 bp, Artificial Sequence
  Description of Artificial Sequence Synthetic primer
tgaattcaaggaggcgtttct

SEQ ID NO: 2 - DNA, 21 bp, Artificial Sequence
  Description of Artificial Sequence Synthetic primer
cagggcttccacttcttcttc

SEQ ID NO: 3 - DNA, 21 bp, Artificial Sequence
  Description of Artificial Sequence Synthetic primer
aagcgcatggagaaggacctc

SEQ ID NO: 4 - DNA, 19 bp, Artificial Sequence
  Description of Artificial Sequence Synthetic primer
ggatgtagccgccgaagtg

SEQ ID NO: 5 - DNA, 19 bp, Artificial Sequence
  Description of Artificial Sequence Synthetic primer
ggtggccagtgaggaggaa

SEQ ID NO: 6 - DNA, 20 bp, Artificial Sequence
  Description of Artificial Sequence Synthetic primer
tgctcgcccagacatctctt

SEQ ID NO: 7 - DNA, 20 bp, Artificial Sequence
  Description of Artificial Sequence Synthetic primer
ctctcggcgctgctgctact

SEQ ID NO: 8 - DNA, 20 bp, Artificial Sequence
  Description of Artificial Sequence Synthetic primer
cgctgctggtccttcctctg

SEQ ID NO: 9 - DNA, 26 bp, Artificial Sequence
  Description of Artificial Sequence Synthetic primer
cagcatggccagcgacgagttcaaga

SEQ ID NO: 10 - DNA, 23 bp, Artificial Sequence
  Description of Artificial Sequence Synthetic primer
tgtcgtcggcatccaggtcatac

SEQ ID NO: 11 - DNA, 18 bp, Artificial Sequence
  Description of Artificial Sequence Synthetic primer
  modified_base (17): a, c, g, t, unknown or other
gcagcatgtgggarctnm

SEQ ID NO: 12 - DNA, 23 bp, Artificial Sequence
  Description of Artificial Sequence Synthetic primer
  modified_base (6): inosine
  modified_base (12): inosine
  modified_base (15): a, c, g, t, unknown or other
  modified_base (18): a, c, g, t, unknown or other
ctyacngcrttnacngcnagrtc

SEQ ID NO: 13 - DNA, 17 bp, Artificial Sequence
  Description of Artificial Sequence Synthetic primer
aactgctccgccaccaa

SEQ ID NO: 14 - DNA, 20 bp, Artificial Sequence
  Description of Artificial Sequence Synthetic primer
ctcacagtgccttccgtctc

(19) United States
(12) Patent Application Publication
(10) Pub. No.: US 2007/0059293 A1
(43) Pub. Date: Mar. 15, 2007

(54) Tissue engineered construct for supplementing or replacing a damaged organ

(75) Inventor: Anthony Atala, Winston Salem, NC (US)

Correspondence Address: DAVID S. RESNICK, 100 SUMMER STREET, NIXON PEABODY LLP, BOSTON, MA, 02110-2131, US

(73) Assignee: Children's Medical Center Corporation, Boston, MA (US)

(21) Appl. No.: 11/507,402

(22) Filed: Aug. 21, 2006

Related U.S. Application Data
(63) Continuation of application No. 10/358,077, filed on Feb. 4, 2003.
(60) Provisional application No. 60/355,504, filed on Feb. 5, 2002.
(60) Provisional application No. 60/364,863, filed on Mar. 15, 2002.

(51) Int. Cl. A61K 35/14; C12N 5/08

(52) U.S. Cl. 424/93.7; 435/366

(57) ABSTRACT

The present invention provides methods and compositions for supplementing or replacing a damaged organ. The damaged organ to be supplemented or replaced in accordance with the present invention include, for example, kidney, heart, liver, spleen, pancreas, bladder, ureter and urethra. In one embodiment, the tissue-engineered construct of the invention has has at least the following characteristics: (a) differentiated cells on a three-dimensional biocompatible scaffold, wherein the differentiated cells originated from transferred pluripotent cells; and (b) at least one physiological function of the organ.

### FIELD OF THE INVENTION

The present invention relates to methods of constructing and using tissue engineered constructs with at least on physiological function to supplement or replace a damaged organ.

### BACKGROUND OF THE INVENTION

It has been estimated that by 2010 more than two million patients will suffer from end-stage renal disease, at an aggregate cost of more than $1 trillion during the coming decade 13. Because of its complex structure and function 14, the kidney is one of the most challenging organs in the body to reconstruct. Previous efforts in kidney tissue engineering have been directed toward the development of an extracorporeal renal support system comprising both biologic and synthetic components 15-17. This approach was first described by Aebischer et al. 18, 19 and is now being focused toward the treatment of acute rather than chronic renal failure. Humes et al. 15 have shown that the combination of hemofiltration and a renal-assist device containing tubule cells can replace certain physiologic functions of the kidney when the filter and device are connected in an extravascular-perfusion circuit in uremic dogs. Heat exchangers, flow and pressure monitors, and multiple pumps are required for optimal functioning of this device 20, 21. Although ex vivo organ substitution therapy would be life-sustaining, there would be obvious benefits for patients if such devices could be implanted on a long-term basis without the need for an extracorporeal-perfusion circuit or immunosuppressive drugs and/or immunomodulatory protocols.

### SUMMARY OF THE INVENTION

The present inventor has developed a tissue-engineered renal construct that avoids the problems seen with prior ex vivo organ substitution therapy. As illustrated in the Example, the cells of the tissue-engineered renal construct organized themselves into glomeruli- and tubule-like structures and exhibited the physiological renal function of excreting metabolic waste products through a urinelike fluid.

The present invention provides methods and compositions for supplementing or replacing a damaged organ. The damaged organ to be supplemented or replaced in accordance with the present invention include, for example, kidney, heart, liver, spleen, pancreas, bladder, ureter and urethra.

In one embodiment, the tissue-engineered construct of the invention has has at least the following characteristics:
(a) differentiated cells on a three-dimensional biocompatible scaffold, wherein the differentiated cells originated from transferred pluripotent cells; and
(b) at least one physiological function of the organ.

Preferably, the construct exhibits about 2% of at least one physiological function of a native healthy organ of similar volume, preferably about 5%, more preferably about 10%. For example, in one embodiment when the construct is selected to supplement or replace the activity of a kidney, the preferred physiological function of the construct is the excretion of metabolic waste. In another embodiment, when the construct is a liver, the preferred physiological function of the organ is secretion of liver specific enzymes, for example ALT, or metabolizing bilirubin. In a further embodiment, when the construct is a pancreas, the preferred physiological function of the organ is the production of insulin. In yet another embodiment, when the construct is a spleen, the preferred physiological function is generation of tuftsin. If necessary, more than one construct may be used to supplement or replace a damaged organ.

The biocompatible scaffold can be formed from a polymer, hydrogel or decellularized tissue.

In one embodiment, the pluripotent cells are differentiated to result in a desired cell type prior to contact with the scaffold. In an alternative embodiment, the pluripotent cells are differentiated after contact with the scaffold. The cells may be differentiated in vitro or in vivo.

Preferably, the pluripotent cells are human stem cells. Preferred human stem cells include, for example, pluripotent hematopoietic stem cells, embryonic stem cells and adult somatic stem cells. The pluripotent cells can be obtained from any suitable tissues including, for example, bone marrow, muscle, adipose tissue, liver, heart, lung and nervous system. The tissue may be adult, embryonic or fetal.

The tissue-engineered construct can be constructed to allow it to function in the host like the organ it was designed to replace or supplement. For example, when the construct is designed to supplement or replace the activity of a kidney, the construct is designed to include a portion that function like a ureter, i.e., conveys urine to from the kidney to the bladder. The “ureter-like” function may be accomplished using a tissue engineered construct or by use of a biocompatible device. Such a device can include a porous membrane structure having an external surface defining an enclosed internal space, e.g., a tube, having at least one effluent channel extending from the construct. Upon implantation, the effluent channel is surgically connected to the bladder.

The invention further provides a method of producing a tissue-engineered construct for supplementing or replacing a damaged organ. The method comprises:
(a) contacting pluripotent cells with a three-dimensional biocompatible scaffold such that the cells attach to the scaffold;
(b) placing the pluripotent cells under conditions to result in differentiation to a desired cell type; and
(c) culturing the cells attached to the scaffold to produce a tissue layer having at least one physiological function of the organ, thereby producing a tissue-engineered construct.

In an alternative embodiment, the method of producing a tissue-engineered construct comprises:
(a) contacting differentiated cells with a three-dimensional biocompatible scaffold such that the cell attach to the scaffold, wherein the differentiated cells originated from transferred pluripotent cells and said pluripotent cells were placed under conditions that caused differentiation; and
(b) culturing the cells attached to the scaffold to produce a tissue layer having at least one physiological function of the organ, thereby producing a tissue-engineered construct.

The present invention further provides a method for supplementing or replacing a damaged organ comprising implanting the tissue-engineered construct of the invention into a host in need thereof.

### BRIEF DESCRIPTION OF FIGURES

The file of this patent contains one drawing executed in color. Copies of this patent with the color drawing will be provided by the Patent and Trademark Office upon request and payment of the necessary fee.

FIGS. 1A-1J show retrieved muscle tissues. (Fig. A) Retrieved cloned cardiac tissue shows a well-organized cellular orientation six weeks after implantation. (Fig. B) Immunocytochemical analysis using troponin I antibodies (brown) identifies cardiac fibers within the implanted constructs six weeks after implantation. (Fig. C) Cardiac cell implant in control group shows fibrosis and necrotic debris (d) at six weeks. (Fig. D) Cloned skeletal muscle cell implants show well-organized bundle formation (12 weeks). (Fig. E) Retrieved skeletal cell implant with polymer fibers (arrows) at 12 weeks. (Fig. F) Immunohistochemical analysis using sarcomeric tropomyosin antibodies (brown) identifies skeletal fibers within the implanted second-set constructs 12 weeks after implantation. (Fig. G) Retrieved cloned skeletal cell implants show spatially oriented muscle fiber 12 weeks after implantation. (Figs. H, I) Retrieved control skeletal cell implants show fibrosis with increased inflammatory reaction (arrows) and necrotic debris at 12 weeks. (Fig. J) Immunocytochemical analysis using CD4 antibodies (brown) identifies CD4⁺ T cells within the implanted control cardiac construct six weeks after implantation. Bars, 100 μm (Figs. A, B, E); 200 μm (Figs. C, G, I, J); 800 μm (Figs. D, F, H). Panels (Figs. A, C-E, G-I), H&E staining.

FIGS. 2A-2D show RT-PCR and western blot analyses. Semi-quantitative RT-PCR products indicate specific mRNA in the retrieved skeletal muscle tissue (Fig. A) and cardiac muscle tissue (Fig. B). Western blot analysis of the implants confirmed the expression of specific proteins in the skeletal muscle tissues (Fig. C) and cardiac muscle tissues (Fig. D). CL6 and CL12, cloned group at 6 and 12 weeks, respectively; CO6 and CO12, control group at 6 and 12 weeks, respectively.

FIGS. 3A-3D show tissue-engineered renal units. (Fig. A) Illustration of renal unit and units retrieved three months after implantation. (Fig. B) Unseeded control. (Fig. C) Seeded with allogeneic control cells. (Fig. D) Seeded with cloned cells, showing the accumulation of urinelike fluid.

FIGS. 4A-4G demonstrate characterization of renal explants. (Figs. A, B) Cloned cells stained positively with synaptopodin antibody (green; Fig. A) and AQP1 antibody (green; Fig. B). (Fig. C) The allogeneic controls displayed a foreign-body reaction with necrosis. (Fig. D) Cloned explant shows organized glomeruli-like structures. Vascular tufts (v); visceral epithelium (arrow). H&E. (E) Organized tubules (arrows) were shown in the retrieved cloned explant. (Fig. F) Immunohistochemical analysis using Factor VIII antibodies (brown) identifies vascular structures. (Fig. G) There was a clear unidirectional continuity between the mature glomeruli, their tubules, and the polycarbonate membrane. Bars, 100 μm (Figs. B, D-F); 200 μm (Fig. A); 800 μm (Fig. C).

FIG. 5 shows RT-PCR analyses (top panel) confirming the transcription of AQP1, AQP2, Tamm-Horsfall, and synaptopodin genes exclusively in the cloned group (Cls). Western blot analysis (bottom panel) confirms high protein levels of AQP1 and AQP2 in the cloned group, whereas expression intensities of CD4 and CD8 were significantly higher in the unseeded and allogeneic control groups (Co 1 and Co 2, respectively). Each lane represents a different cloned tissue.

FIG. 6 shows an Elispot analyses of the frequencies of T cells that secrete IFN-γ after primary and secondary stimulation with allogeneic renal cells, cloned renal cells, or nuclear donor fibroblasts. The presented wells are single representatives of the duplicate wells for each responder-stimulator combination.

FIG. 7 shows results of a chemical analysis of fluids produced by the renal units (Table 1).

FIG. 8 shows nucleotide and amino acid substitutions distinguishing nuclear donor and cloned cells (Table 2).

### DETAILED DESCRIPTION OF THE INVENTION

The present invention provides methods and compositions for supplementing or replacing a damaged organ. The damaged organ to be supplemented or replaced in accordance with the present invention include, for example, kidney, heart, liver, spleen, pancreas, bladder, ureter and urethra.

In one embodiment, the tissue-engineered construct of the invention has at least the following characteristics:
(a) differentiated cells on a three-dimensional biocompatible scaffold, wherein the differentiated cells originated from transferred pluripotent cells; and
(b) at least one physiological function of the organ.

The phrase “supplementing a damaged organ” as used herein refers to increasing, enhancing, improving, the function of an organ that is operating at less than optimum capacity. The term is used to refer to a gain in function so that the organ is operating at a physiologically acceptable capacity for that subject. For example, the physiological acceptable capacity for an organ from a child, e.g., a kidney or heart, would be different from the physiological acceptable capacity of an adult, or an elderly patient. The entire organ, or part of the organ can be supplemented. Preferably the supplementation results in an organ with the same physiological response as a native organ. In a preferred embodiment, an organ is supplemented in capacity when it is functioning to at least at about 10% of its natural capacity.

When the three-dimensional biocompatible scaffold after contact with pluripotent cells are brought into contact with a host tissue at a target site (e.g., within the organ), it is able to grow and proliferate within the target site and replace or supplement the depleted activity of the organ. The construct can be added at a single location in the host. Alternatively, a plurality of constructs can be created and added to multiple sites in the host.

The term “target site” as used herein refers to region in the host or organ that requires replacement or supplementation. The target site can be a single region in the organ or host, or can be multiple regions in the organ or host. Preferably the supplementation or replacement results in the same physiological response as a normal organ.

The shape and dimensions of the scaffold is determined based on the organ being replaced or supplemented, and the type of scaffold material being used to create the construct. For example, if a polymeric scaffold is used for kidney replacement or supplementation, the dimension of the polymeric scaffold can vary in terms of width and length of the polymeric scaffold. The skilled artisan will appreciate that the size and dimensions of the polymeric scaffold will be determined based on the area of the organ being replaced or supplemented.

The term “decellularized” or “decellularization” as used herein refers to a biostructure (e.g., an organ, or part of an organ), from which the cellular and tissue content has been removed leaving behind an intact acellular infra-structure. Organs such as the kidney are composed of various specialized tissues. The specialized tissue structures of an organ, or parenchyma, provide the specific function associated with the organ. The supporting fibrous network of the organ is the stroma. Most organs have a stromal framework composed of unspecialized connecting tissue which supports the specialized tissue. The process of decellularization removes the specialized tissue, leaving behind the complex three-dimensional network of connective tissue. The connective tissue infra-structure is primarily composed of collagen. The decellularized structure provides a matrix material onto which different cell populations can be infused. Decellularized biostructures can be rigid, or semi-rigid, having an ability to alter their shapes. Examples of decellularized organs useful in the present invention include, but are not limited to, the heart, kidney, liver, pancreas, spleen, bladder, ureter and urethra. Culture and construction of decellularized biostructures can be performed, for example, as describe in U.S. Pat. No. 6,479,064, which is herein incorporated by reference in its entirety.

The terms “subject” or “host,” as used herein, includes, but is not limited to, humans, nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be covered.

Isolated pluripotent cells can be cultured in vitro to increase the number of cells available for coating the scaffold. The use of allogenic cells, and more preferably autologous cells, is preferred to prevent tissue rejection. However, if an immunological response does occur in the subject after implantation of the artificial organ, the subject may be treated with immunosuppressive agents such as, cyclosporin or FK506, to reduce the likelihood of rejection.

Preferred pluripotent cells are stem cells. Stem cells can be derived from a human donor, e.g., pluripotent hematopoietic stem cells, embryonic stem cells, adult somatic stem cells, and the like. Stem cells can also be obtained from amniotic fluid, chorionic villus and placenta. See, PCT/US02/36966 which is incorporated herein as a reference by its entirety. The stem cells can be cultured in the presence of combinations of polypeptides, recombinant human growth and maturation promoting factors, such as cytokines, lymphokines, colony stimulating factors, mitogens, growth factors, and maturation factors, so as to differentiate into the desired cells type, e.g., renal cells, or cardiac cells. Method for stem cell differentiation into kidney and liver cells from adult bone marrow stem cells (BMSCs) are described for example by Forbes et al. (2002) Gene Ther 9:625-30. Protocols for the in vitro differentiation of embryonic stem cells into cells such as cardiomyocytes, representing all specialized cell types of the heart, such as atrial-like, ventricular-like, sinus nodal-like, and Purkinje-like cells, have been established (See e.g., Boheler et al (2002) Circ Res 91:189-201). Further examples of differentiation-inducing agents and combinations thereof for differentiating desired cell lineages can be found at Stem Cells: Scientific Progress and Future Research Directions. (Appendix D. Department of Health and Human Services. June 2001. http://www.nih.gov/news/stemcell/scireport.htm).

For example, mesodermal cell differentiation was achieved from blastocyst innercell mass (H9 clone line from Thomson et al. (Science, 1998, 282:1145-1147) using basic fibroblast growth factor, transforming growth factor beta 1, activin-A, bone morphogenic protein 4, hepatocyte growth factor, epidermal growth factor beta, nerve growth factor and retinoic acid as described in Schuldiner et al. (Proc. Natl. Acad. Sci. U.S.A., 2000, 97:11307-11312). Mesodermal cells grown under aforementioned conditions were shown to give raise to muscle, bone, kidney, urogenital, heart, and hematopoietic cells. Pancreatic beta cells can be differentiated using conditions described for embryoid body formation as detailed in Itskovich-Eldor et al. (Mol. Med. 2000, 6:88-95), but without the addition of leukemia inhibitory factor or basic fibroblast growth factor as described in detail in Assady et al. (2001, Diabetes 50, http://diabetes.org/Diabetes Rapids/Suheir Assady 0682001.pdf).

Multipotent stem cells from metanephric mesenchyme can generate at least three distinct cell types; glomerular, proximal and distal epithelia, i.e., differentiation into a single nephron segment (See e.g., Herzlinger et al. (1992) Development 114:565-72). Multipotent cells can also be isolated during different developmental stages. For example, isolation of kidney cells from fetal or neonatal tissues is described in detail in WO 98/09582, which is herein incorporated by reference in its entirety.

In addition to embryonic stem cells, adult stem cells can give raise to cell useful according to the present invention. For example, hematopoietic stem cells can be differentiated into hepatocytes by exposing them to bone marrow (Allison et al. Nature, 2000, 406:257 and Theise et al. Hepatology, 2000, 32:11-16). Further, nestin positive islet-derived progenitor cells can be differentiated into pancreatic and hepatic cells when cultured for extended periods as described in Zulewski et al. (Diabetes, 2001, 50:521-533).

The tissue-engineered constructs of the present invention are created using scaffold materials as the substrate onto which cells are deposited, and on which cells grown and adhere. It is important to recreate, in culture, the cellular microenvironment found in vivo for the particular organ targeted for replacement or supplementation. Retaining an infra-structure that is similar or the same as an in vivo organ creates the optimum environment for cell-cell interactions, development and differentiation of cell populations.

The invention provides a method of forming tissue-engineered constructs using a scaffold material that supports the maturation, development and differentiation, of additional cultured cells in vitro to form components of adult tissues analogous to their in vivo counterparts. The scaffold allows optimum cell-cell interactions, thereby allowing a more natural formation of cellular phenotypes and a tissue microenvironment. The scaffold also allows cells to continue to grow actively, proliferate and differentiate to produce a tissue engineered construct that is also capable of supporting the growth, proliferation and differentiation of additional cultured cells populations, if needed.

Cells grown on the scaffold materials, in accordance with the present invention, may grow in multiple layers, forming a cellular structure that resembles physiologic conditions found in vivo. The scaffold can support the proliferation of different types of cells and the formation of a number of different tissues. Examples include, but are not limited to, kidney, heart, skin, liver, pancreas, adrenal and neurological tissue, as well as tissues of the gastrointestinal and genitourinary tracts, and the circulatory system.

The seeded scaffold can be used in a variety of applications. For example, the scaffold can be implanted into a subject. Implants, according to the invention, can be used to replace or supplement existing tissue. For example, to treat a subject with a kidney disorder by replacing or supplementing the natural kidney. The subject can be monitored after implantation for amelioration of the kidney disorder.

In a preferred embodiment, the scaffold is a polymeric material. Examples of suitable polymers include, but are not limited to, collagen, poly(alpha esters) such as poly(lactate acid), poly(glycolic acid), polyorthoesters and polyanhydrides and their copolymers, cellulose ether, cellulose, cellulosic ester, fluorinated polyethylene, phenolic, poly-4-methylpentene, polyacrylonitrile, polyamide, polyamideimide, polyacrylate, polybenzoxazole, polycarbonate, polycyanoarylether, polyestercarbonate, polyether, polyetheretherketone, polyetherimide, polyetherketone, polyethersulfone, polyethylene, polyfluoroolefin, polylmide, polyolefin, polyoxadiazole, polyphenylene oxide, polyphenylene, sulfide, polypropylene, polystyrene, polysulfide, polysulfone, polytetrafluoroethylene, polythioether, polytriazole, polyurethane, polyvinylidene fluoride, regenerated cellulose, urea-formaldehyde, or copolymers or physical blends of these materials.

Polymers, such as polyglycolic acid, which is a suitable biocompatible structures for producing an organ augmenting structure. The biocompatible polymer may be shaped using methods such as, solvent casting, compression molding, filament drawing, meshing, leaching, weaving and coating.

Other scaffold materials include biodegradable polymers including polyglycolic and acid polymers (PGA), polylactic acid polymers (PLA), polysebacic acid polymers (PSA), poly(lactic-co-glycolic) acid copolymers (PLGA), poly(lactic-co-sebacic) acid copolymers (PLSA), poly(glycolic-co-sebacid) acid copolymers (PGSA), and polyhydroxyalkanoate (PHA). PHAs and their production are described in, for example, PCT Publication Nos. WO99/14313, WO99/32536 and W000/56376. Combinations of biodegradable polymers, e.g., PGA and PLGA, can also be used.

Other biodegradable polymers useful in the present invention include polymers or copolymers of caprolactones, carbonates, amides, amino acids, orthoesters, acetals, cyanoacrylates and degradable urethanes, as well as copolymers of these with straight chain or branched, substituted or unsubstituted, alkanyl, haloalkyl, thioalkyl, aminoalkyl, alkenyl, or aromatic hydroxy- or di-carboxylic acids. In addition, the biologically important amino acids with reactive side chain groups, such as lysine, arginine, aspartic acid, glutamic acid, serine, threonine, tyrosine and cysteine, or their enantiomers, may be included in copolymers with any of the aforementioned materials.

Since this invention employs cell seeded scaffolds for preparing therapeutic tissues, it is necessary for the scaffold to be biocompatible and conducive to cell attachment and subsequent tissue growth. It is therefore desirable to be able to adjust surface properties to suit the intended application, without altering other properties of the scaffold such as its mechanical strength or thermal properties. Useful surface modifications could include, for example, changes in chemical group functionality, surface charge, hydrophobicity, hydrophilicity, and wettability. For example, it would be desirable to improve or maximize cellular attachment or allow for the attachment of the desired cell type or types. This can be accomplished, for example, by attaching or coating the surface with a bioactive compound or peptide which promotes cellular attachment. The coating or bioactive compound may be attached to the surface either covalently or non-covalently. Such skills are well known in the art.

Sterilization is performed prior to seeding the scaffold with cells. Heat sterilization is often impractical since the heat treatment could deform the device, especially if the materials have a melting temperature below that required for the heat sterilization treatment. This problem can be overcome using cold ethylene oxide gas as a sterilizing agent.

Suitable growth conditions and media for cells in culture are well known in the art. Cell culture media typically comprise essential nutrients, but also optionally include additional elements (e.g., growth factors, salts and minerals) which may be customized for the growth and differentiation of particular cell types.

The polymeric matrix can be fabricated to have a controlled pore structure that allows nutrients from the culture medium to reach the deposited cell population, but prevent cultured cells from migrating through the pores. In vitro cell attachment and cell viability can be assessed using scanning electron microscopy, histology and quantitative assessment with radioisotopes.

The polymeric matrix can be shaped into any number of desirable configurations to satisfy any number of overall system, geometry or space restrictions. The polymeric matrix can be shaped to different sizes to conform to the organs of different sized patients. The polymeric matrix may also be shaped to facilitate special needs of a patient, for example, a disabled patient, who may have a different abdominal cavity space may require an organ or part of an organ reconstructed to adapt to fit the space.

In other embodiments, the polymeric matrix is used for the treatment of laminar structures in the body such as urethra, vas deferens, fallopian tubes, lacrimal ducts. In those applications the polymeric substrate can be shaped as a hollow tube.

The tissue-engineered construct can be flat, tubular, or of complex geometry. The shape of the construct will be decided by its intended use. The construct can be implanted to repair, supplement, or replace diseased or damaged parts of organs.

In one embodiment, the scaffold material is a hydrogel composed of crosslinked polymer networks which are typically insoluble or poorly soluble in water, but can swell to an equilibrium size in the presence of excess water. For example, the cells can be placed in a hydrogel and the hydrogel injected into desired locations within the organ. In one embodiment, the cells can be injected with collagen alone. In another embodiment, the cells can be injected with collagen and other hydrogels. The hydrogel compositions can include, without limitation, for example, poly(esters), poly(hydroxy acids), poly(lactones), poly(amides), poly(ester-amides), poly(amino acids), poly(anhydrides), poly(ortho-esters), poly(carbonates), poly(phosphazines), poly(thioesters), polysaccharides and mixtures thereof. Furthermore, the compositions can also include, for example, a poly(hydroxy) acid including poly(alpha-hydroxy) acids and poly(beta-hydroxy) acids Such poly(hydroxy) acids include, for example, polylactic acid, polyglycolic acid, polycaproic acid, polybutyric acid, polyvaleric acid, and copolymers and mixtures thereof. Due to the unique properties of hydrogels and their potential applications in such areas as controlled drug delivery, various types of hydrogels have been synthesized and characterized. Most of this work has focused on lightly cross-linked, homogeneous homopolymers and copolymers.

The bulk polymerization, i.e., polymerization in the absence of added solvent, of monomers to make a homogeneous hydrogel produces a glassy, transparent polymer scaffold which is very hard. When immersed in water, the glassy matrix swells to become soft and flexible. Porous hydrogels are usually prepared by a solution polymerization technique, which entails polymerizing monomers in a suitable solvent. The nature of a synthesized hydrogel, whether a compact gel or a loose polymer network, depends on the type of monomer, the amount of diluent in the monomer mixture, and the amount of crosslinking agent. As the amount of diluent (usually water) in the monomer mixture increases, the pore size also increases up to the micron range. Hydrogels with effective pore sizes in the 10-100 run range and in the 100 nm-10 micrometer range are termed “microporous” and “macroporous” hydrogels, respectively. The microporous and macroporous structures of hydrogels can be distinguished from those of non-hydrogel porous materials, such as porous polyurethane foams. In the plastic foam area, micro- and macro-pores are indicated as having pores less than 50 micrometers and pores in the 100-300 micrometer range, respectively. One of the reasons for this difference is that hydrogels with pores larger than 10 micrometers are uncommon, while porous plastics having pores in the 100-300 micrometer range are very common.

Microporous and macroporous hydrogels are of ten called polymer “sponges.” When a monomer, e.g., hydroxyethyl methacrylate (HEMA), is polymerized at an initial monomer concentration of 45 (w/w) % or higher in water, a hydrogel is produced with a porosity higher than the homogeneous hydrogels. The matrix materials of present invention encompass both conventional foam or sponge materials and the so-called “hydrogel sponges.” For a further description of hydrogels, see U.S. Pat. No. 5,451,613 (issued to Smith et al.).

In another embodiment, the scaffold is created using parts of a natural decellularized organ. Parts of organs can be decellularized by removing the entire cellular and tissue content from the organ. The decellularization process comprises a series of sequential extractions. One key feature of this extraction process is that harsh extraction that may disturb or destroy the complex infra-structure of the biostructure, be avoided. The first step involves removal of cellular debris and solubilization of the cell membrane. This is followed by solubilization of the nuclear cytoplasmic components an the nuclear components. See, for example, U.S. Pat. No. 6,479,064.

Preferably, the biostructure, e.g., part of an organ is decellularized by removing the cell membrane and cellular debris surrounding the part of the organ using gentle mechanical disruption methods. The gentle mechanical disruption methods must be sufficient to disrupt the cellular membrane. However, the process of decellularization should avoid damage or disturbance of the biostructure's complex infra-structure. Gentle mechanical disruption methods include scraping the surface of the organ part, agitating the organ part, or stirring the organ in a suitable volume of fluid, e.g., distilled water. In one preferred embodiment, the gentle mechanical disruption method includes stirring the organ part in a suitable volume of distilled water until the cell membrane is disrupted and the cellular debris has been removed from the organ.

After the cell membrane has been removed, the nuclear and cytoplasmic components of the biostructure are removed. This can be performed by solubilizing the cellular and nuclear components without disrupting the infra-structure. To solubilize the nuclear components, non-ionic detergents or surfactants may be used. Examples of non-ionic detergents or surfactants include, but are not limited to, the Triton series, available from Robin and Haas of Philadelphia, Pa., which includes Triton X-100, Triton N-101, Triton X-114, Triton X-405, Triton X-705, and Triton DF-16, available commercially from many vendors; the Tween series, such as monolaurate (Tween 20), monopalmitate (Tween 40), monooleate (Tween 80), and polyoxethylene-23-lauryl ether (Brij. 35), polyoxyethylene ether W-1 (Polyox), and the like, sodium cholate, deoxycholates, CHAPS, saponin, n-Decyl-D-glucopuranoside, n-heptyl-D glucopyranoside, n-Octyl-D-glucopyranoside and Nonidet P-40.

One skilled in the art will appreciate that a description of compounds belonging to the foregoing classifications, and vendors may be commercially obtained and may be found in “Chemical Classification, Emulsifiers and Detergents”, McCutcheon's, Emulsifiers and Detergents, 1986, North American and International Editions, McCutcheon Division, MC Publishing Co., Glen Rock, N.J., U.S.A. and Judith Neugebauer, A Guide to the Properties and Uses of Detergents in Biology and Biochemistry, Calbiochem. R., Hoechst Celanese Corp., 1987. In one preferred embodiment, the non-ionic surfactant is the Triton. series, preferably, Triton X-100.

The concentration of the non-ionic detergent may be altered depending on the type of biostructure being decellularized. For example, for delicate tissues, e.g., blood vessels, the concentration of the detergent should be decreased. Preferred concentrations ranges non-ionic detergent can be from about 0.001 to about 2.0% (w/v). More preferably, about 0.05 to about 1.0% (w/v). Even more preferably, about, 0.1% (w/v) to about 0.8% (w/v). Preferred concentrations of these range from about 0.001 to about 0.2% (w/v), with about 0.05 to about 0.1% (w/v) particular preferred.

The cytoskeletal component, comprising consisting of the dense cytoplasmic filament networks, intercellular complexes and apical microcellular structures, may be solubilized using alkaline solution, such as, ammonium hydroxide. Other alkaline solution consisting of ammonium salts or their derivatives may also be used to solubilize the cytoskeletal components. Examples of other suitable ammonium solutions include ammonium sulphate, ammonium acetate and ammonium hydroxide. In a preferred embodiment, ammonium hydroxide is used.

The concentration of the alkaline solutions, e.g., ammonium hydroxide, may be altered depending on the type of biostructure being decellularized. For example, for delicate tissues, e.g., blood vessels, the concentration of the detergent should be decreased. Preferred concentrations ranges can be from about 0.001 to about 2.0% (w/v). More preferably, about 0.005 to about 0.1% (w/v). Even more preferably, about, 0.01% (w/v) to about 0.08% (w/v).

The decellularized, lyophilized structure may be stored at a suitable temperature until required for use. Prior to use, the decellularized structure can be equilibrated in suitable isotonic buffer or cell culture medium. Suitable buffers include, but are not limited to, phosphate buffered saline (PBS), saline, MOPS, HEPES, Hank's Balanced Salt Solution, and the like. Suitable cell culture medium includes, but is not limited to, RPMI 1640, Fisher's, Iscove's, McCoy's, Dulbecco's medium, and the like.

In some embodiments, attachment of the cells to the matrix material is enhanced by coating the matrix material with compounds such as basement membrane components, agar, agarose, gelatin, gum arabic, collagens types I, II, III, IV, and V, fibronectin, laminin, glycosaminoglycans, mixtures thereof, and other hydrophilic and peptide attachment materials known to those skilled in the art of cell culture. A preferred material for coating the scaffold material is collagen.

In other embodiments, scaffold materials can be treated with factors or drugs prior to implantation, before or after the matrix material is coated with cultured cells, e.g., to promote the formation of new tissue after implantation. Factors including drugs, can be incorporated into the matrix material or be provided in conjunction with the matrix material. Such factors will in general be selected according to the tissue or organ being reconstructed or augmented, to ensure that appropriate new tissue is formed in the engrafted organ or tissue (for examples of such additives for use in promoting bone healing, (see, e.g., Kirker-Head, (1995) Vet. Surg. 24: 408-19). For example, when matrix materials are used to augment vascular tissue, vascular endothelial growth factor (VEGF), can be employed to promote the formation of new vascular tissue (see, e.g., U.S. Pat. No. 5,654,273 issued to Gallo et al.). Other useful additives include antibacterial agents such as antibiotics.

Cells perfused onto the scaffold can be incubated to allow the cells to adhere. The adhered cells can be cultured in vitro in culture medium to allow the cells to grow and develop until the cells resemble a morphology and structure similar to that of the organ to be supplemented or replaced. The cells can be differentiated before or after perfusion onto the scaffold.

Alternatively, after perfusing the cells, the scaffold can be implanted in vivo without prior in vitro culturing of the cells. The cells chosen for perfusion will depend upon the organ being augmented. For example, construction of a kidney-like tissue construct will involve infusing pluripotent cells onto the scaffold. The cells are placed under conditions to result in a renal cell either before or after perfusion or contact with the scaffold. The cells are then cultured until they differentiate into kidney-like tissue and exhibit at least one physiological function of a kidney tissue, e.g., excretion of metabolic waste. The physiological function is selected according to the desired organ. For example, one physiological function of a liver is production of liver cell specific enzymes including alanine aminotransferase (ALT, the normal range of ALT levels is between 5 IU/L to 60 IU/L (International Units per Liter)) and aspartate aminotransferase (AST, the normal range for AST levels in the bloodstream are about 5 IU/L to 43 IU/L.). Alternatively, one can measure bilirubin (unmetabolized bilirubin)/direct bilirubin (metabolized bilirubin) ratio which reflects capacity of the liver to metabolize bilirubin. Direct bilirubin testing measures bilirubin made in the liver. The normal level of direct bilirubin ranges from about 0.1 to about 0.3 mg/dL of blood. A physiological function of a spleen can be, for example, generation of tuftsin, a splenic endocarboxipeptidase (Nishioka et al. 1972, Zvi et al. 1997). The pancreas plays an important role in regulating body chemistry by releasing some of the enzymes used in the digestion of food as well as glucagen and insulin. Measurement of these enzymes and insulin levels produced by the pancreatic islet cells allows assessment of the physiological function of a spleen organ construct. Motor and sensory nerves are essential for the function of bladder. Nerve function can be measured using electromyograpgy (see, e.g. Vodusek Curr Opin Obstet Gynecol. 2002 October; 14(5):509-14).

In another embodiment, the tissue-engineered construct can be used to supplement heart function. In this example, the construct can be created by seeding the scaffold with a population myocardial cells originating from pluripotent cells. The construct is cultured until it exhibits at least one physiological function of a heart tissue, e.g., myocardial contractility. The construct can be used to supplement heart function by implanting the construct at or near an area of the heart that has been damaged or infracted.

Preferably, the construct exhibits about 2% of at least one physiological function of a native healthy organ of similar volume, preferably about 5%, more preferably about 10%.

The tissue-engineered constructs are implanted into a host or host organ using standard surgical procedures. These surgical procedures may vary according to the organ being supplemented or replaced.

The present invention solves the problems of the prior art by providing a method whereby differentiated cells and adult stem cells may be generated in vivo, by exposing nuclear transfer-generated ICM cells and other pluripotent embryonic cells to the appropriate cellular or tissue-type environment to encourage development of said pluripotent cells along a desired path.

Chemical selection can be accomplished in vivo to encourage the formation of cohesive tissues, and may also be used at the isolation and purification stage to separate the cloned cells away from the cells of the host.

For instance, ICM cells can be injected into adult animals (i.e., SCID or nude mice), or more preferably into animal-embryos or fetuses at various stages of development. These cells could also be implanted into different sites to encourage differentiation into certain cell lineages. Injecting/implanting pluripotent stem cells into fetal environments may foster and encourage the cells to differentiate into cell types, such as pancreatic beta cells, that may not occur efficiently or completely in adult animals or in embryoid bodies in vitro.

Starting chemical selection hours or days after injection or implantation of the cells is particularly important for generating human replacement cells and tissues, because this will eliminate the ethical/legal fear that a human baby or other human-animal entity will develop in the host animal. Furthermore, the sooner the growth and differentiation of the human pluripotent cells in a host mammalian embryo or fetus is restricted, the further replacement cell derivation can proceed without disrupting fetal development and growth.

The methods of the invention also encompass a multi-level targeted differentiation approach, whereby cells may first be encouraged to develop along a particular cell lineage path, such as an endodermal, mesodermal or ectodermal cell lineage, by controlled expression of markers specific for a particular lineage path. At another level, specific cell types and tissues may be isolated from lineage specific partially differentiated cells. For instance, cells can be directed down an endodermal path, from which islets may be then be isolated. Such a multi-level approach has the advantage of focusing a majority of the implanted or injected cells in the desired direction, and facilitates purification of cells following differentiation into the desired cell type.

The present invention provides methods for encouraging the development of pluripotent cells along a particular path of differentiation and development by exposing such cells to environmental cues. Preferred pluripotent cells of the present invention are inner cell mass (ICM) cells, wherein such cells include cells derived or isolated from an ICIVI that have partially differentiated although they are still pluripotent. ES cells and other pluripotent cells may also be used. For instance, the invention includes a method of producing replacement cells and/or tissues for a mammal in need of such replacement cells and/or tissues, comprising (a) isolating an embryonic pluripotent cell or cells; (b) introducing into said embryonic pluripotent cell(s) a selectable marker operatively linked to a cell or tissue specific promoter, enhancer or other regulatory genetic element such that said selectable marker is expressed only in the cell or tissue type of interest; (c) permitting said embryonic cell(s) to differentiate into differentiated cells and tissues; and (d) selecting for cells and tissues that express said selectable marker in order to produce replacement cells and/or tissues.

Preferably, the pluripotent cells employed in the present invention are human pluripotent cells. Such cells may be isolated using nuclear transfer of a human somatic cell nucleus into a mammalian enucleated oocyte or other suitable recipient cell using methods known in the art. In this regard, cross species nuclear transfer is disclosed in PCT/US99/04608 and PCT/US00/05434 both of which are herein incorporated by reference in its entirety. Such methods as applied to human embryonic pluripotent cells are useful for generating replacement cells and tissues to be used for transplantation and to treat various diseases, i.e., heart disease, cancer and diabetes to name a few.

Replacement cells and/or tissues are any desired cell type, but are preferably selected from the group consisting of pancreatic beta cells, brain cells, neurons, cardiomyocytes, fibroblasts, skin cells, liver cells, kidney cells and islets. Alternatively, pluripotent cells can be induced to differentiate along certain development pathways, i.e., endodermal lines, mesodermal lines and ectodermal lines. Specific cell types and adult stem cells could then be isolated from a particular line of partially differentiated cells. For instance, islets could be isolated by encouraging further differentiation of an endodermal line of partially differentiated cells. Alternatively, nerve stem cells or hematopoietic stem cells or other adult stem cells could be obtained which have the potential to differentiate into a variety of cell types.

The pluripotent cells of the invention may be placed into a developing mammal at any appropriate age to facilitate directed differentiation and development. The cells are generally placed in the vicinity of the cell or tissue type desired. For instance, to encourage cardiomyocyte development, the cells may be placed into the heart muscle wall of the developing fetus. Cells may be implanted or injected as a mixture of individual cells, or could be arranged onto a synthetic scaffold or other extracellular matrix material using tissue engineering techniques. For instance, cells to be induced to develop into cardiornyocytes can be arranged on a scaffold and patched onto the heart muscle. Similar patches can be constructed for cells implanted into other organs, i.e., the brain or liver. Such an approach provides the advantage that the cells and formed tissues are readily retrievable following differentiation.

Alternatively, some directed development may be performed in vitro in the presence of cells isolated from a mammal. Also, cells may be mixed with a matrigel substance, or other suitable extracellular matrix material which causes cells to aggregate, in order to encourage tissue formation, either for in vivo implantation or in vitro development in the presence of mixed cell types.

Preferably, the cells are inserted into a developing mammalian fetus that has not yet developed self recognition immune function. For example, a developing fetal sheep does not begin to develop self recognition until the age of 60 days (continuing to about 85 days), so it is possible to introduce human cells before about day 55 to 60 and have the animal be tolerized to the implanted human cells. Thereafter, the human cells may differentiate without adverse immune response, even until the end of term, i.e., 145 days. Different points in time for implanting cells may be critical for different cell types and tissues. The criticality of timing is a parameter that may be readily analyzed by the skilled artisan given the present disclosure as a guide.

The invention also includes variations of the method discussed above as would be envisioned by the skilled artisan. For instance, antigens specific to the donor cells could be injected prior to the time period during which self recognition develops in the host fetus in order to tolerize the fetus to the foreign antigens. Then, cells to be encouraged along particular developmental pathways can be injected either during or after the development of self recognition without adverse immune response. This variation is particularly useful for encouraging differentiation into cell types found in organs that do not fully develop until after the period in which self recognition develops, i.e., thymus cells.

According to the methods of the invention, in order to further assist development along a certain path of differentiation, the embryonic pluripotent cells may be transfected with a selectable marker either prior to implantation, or prior to nuclear transfer, in order to help select cells which have differentiated along the proper pathway. The selectable marker may be any marker that may be employed in mammalian cells. For instance, the selectable marker may be selected from the group consisting of aminoglycoside phosphotransferase, puromycin, zeornycin, hygromycin, GLUT-2 and non-antibiotic resistance selectable marker systems. U.S. Pat. No. 6,162,433 discloses nonantibiotic selectable markers suitable for mammalian use, and is herein incorporated by reference in its entirety. A preferred selectable marker is aminoglycoside phosphotransferase, wherein said differentiated cells are selected by administering G418.

Although a developing fetal mammal is the preferred host environment for directing the development and differentiation of pluripotent cells, any mammalian host may be employed, i.e., including adults, embryos, fetuses and embryoid bodies. For instance, the pluripotent cells of the invention may be implanted into an immune comprised host, such as a SCID mouse. In such an instance, the need to implant pre-tolerance is avoided, and implantation may be directed to more fully developed tissue locations. Also, the host environment need not necessarily be completely an in vivo environment. For instance, an embryoid body may be itself maintained in vitro depending on the stage of development sought and the extent of differentiation possible.

In systems where a selectable marker is employed, replacement cells and/or tissues may be purified by chemical selection in vitro or in vivo. A preferred method employs two separate selectable markers, for instance, neomycin and hygromycin, operably linked to promoters that are specifically expressed in an overlapping group of tissues. For instance, by expressing neomycin from a promoter specifically expressed in bone and splenocytes, and by also expressing hygromycin from a promoter specifically expressed in bone and cartilage, one can achieve a stronger selection for bone cells via an overlapping selection mechanism. In such a system, although the promoters are not cell-specific, the combined selection for both markers results in cell specificity.

Alternatively, replacement cells may be purified away from surrounding host cells and tissues, for instance, using immunopurification targeting a cell-specific, species-specific cell surface protein. Antigens employed for purification of the desired cells may also be expressed via one or more exogenous gene construct(s) that are transfected into said primordial cells. Such exogenous genes may be preferentially expressed in the cells or tissues of interest via cell-specific or tissue-specific promoters. For example, the CID4 antigen can be preferentially expressed in pancreatic beta cells by operably linking the gene for the CID4 antigen to an insulin promoter. Because the CID4 antigen is not generally expressed in pancreatic, the seeded donor cells may be purified away from surrounding host cells by immunopurification using anti-CID4 antibodies, or via another purification process that targets the CID4 protein.

The present invention also encompasses the replacement cells and/or tissues produced by the methods disclosed herein, and methods of using the same to treat patients in need of replacement cells and tissues, i.e., via transplantation. The following examples serve to illustrate the disclosed invention, but should not be construed to limit the scope thereof.

### EXAMPLE

Results and Discussion

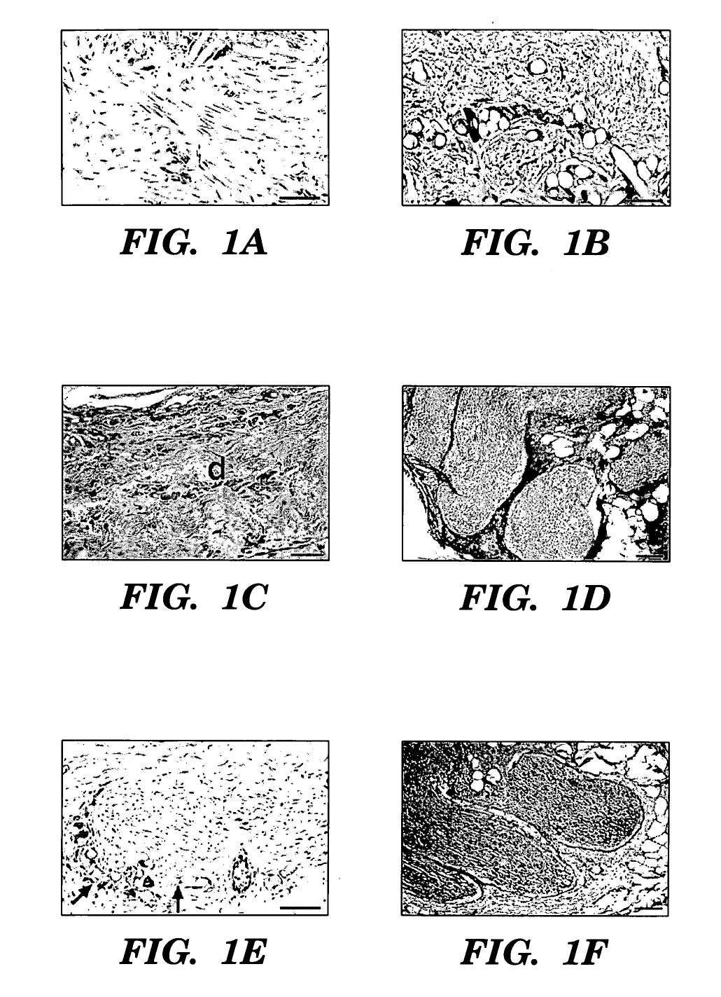

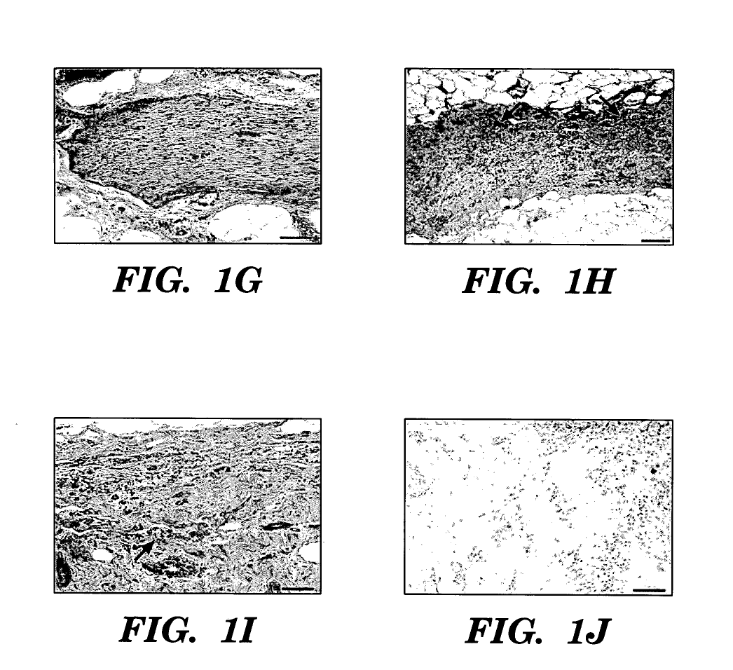

Cardiac and skeletal muscle constructs. Tissue-engineered constructs containing bovine cardiac (n=8) and skeletal muscle cells (n=8) were transplanted subcutaneously and retrieved six weeks after implantation. After retrieval of the first set of implants, a second set of constructs (n=12) from the same donor was transplanted for an additional 12 weeks. On a histologic level, the cloned cardiac tissue appeared intact and showed a well-organized cellular orientation with spindle-shaped nuclei (FIG. 1A). The retrieved tissue stained positively with troponin I antibodies, indicating the preservation of the cardiac muscle phenotype (FIG. 1B). The cloned skeletal cell explants showed spatially oriented tissue bundles with elongated multinuclear muscle fibers (FIGS. 1D, G). Immunohistochemical analysis using sarcomeric tropomyosin antibodies identified skeletal muscle fibers within the implanted constructs (FIG. 1F). In contrast to the cloned implants, the allogeneic control cell implants failed to form muscle bundles, and showed more inflammatory cells, fibrosis, and necrotic debris, consistent with acute rejection (FIGS. 1H, I).

Histologic examination revealed extensive vascularization throughout the implants, as well as the presence of multinucleated giant cells surrounding the remaining polymer fibers. Although nondegraded fibers were present in all tissue specimens, histomorphometric analysis of the explanted tissues indicated that the degree of immune reaction was significantly less in the cloned tissue sections than in the control (66±4 and 54±4 (mean±s.e.m.) total inflammatory cells/high-power field (HPF) for the cloned constructs at 6 weeks (first-set grafts) and 12 weeks (second-set grafts), respectively, vs. 93±3 and 80±3 cells/HPF for the constructs generated from the control cells, P<0.0005; FIG. 1F-G). Immunocytochemical analysis using CD4- and CD8-specific antibodies identified approximately twofold-greater numbers of CD4⁺ and CD8⁺ T cells (13±1.3 and 14±1.4 cells/HPF, respectively, vs. 7±1.1 and 7±1.2 cells/HPF, P<0.00001) within the explanted first- and second-set control as compared with cloned constructs. Notably, cloned constructs from the first and second sets exhibited comparable levels of CD4 and CD8 expression, arguing against the presence of an enhanced second-set reaction as would be expected if mtDNA-encoded minor antigen differences were present.

Polyglycolic acid (PGA) is one of the most widely used synthetic polymers in tissue engineering 28, 29. PGA polymers are biodegradable and biocompatible, and have been used in experimental and clinical settings for decades. Although the scaffolds are accepted by the immune system, PGA is known to stimulate a characteristic pattern of inflammation and ingrowth similar to that observed in the cloned constructs of the present study. However, this response, which is greatest at ˜12 weeks after implantation, can be considered as separate from the immune response to the transplanted cells, although there can clearly be interactions between the two 30-35.

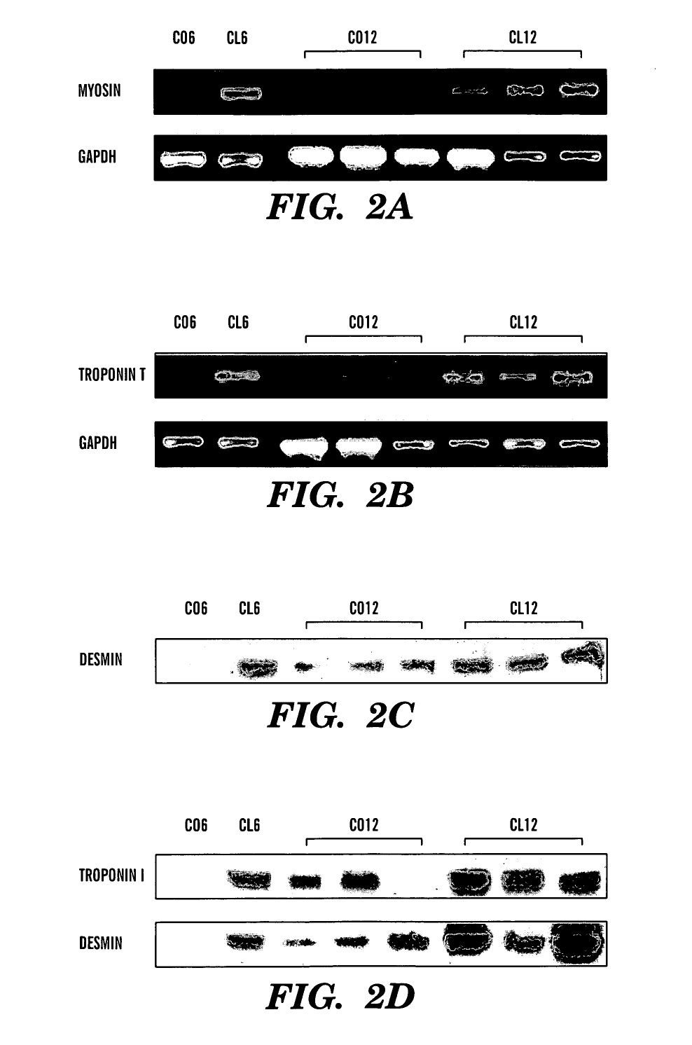

Semiquantitative RT-PCR and western blot analysis confirmed the expression of specific mRNA and proteins in the retrieved tissues despite the presence of allogeneic mitochondria. Mean expression intensities of myosin/GAPDH and troponin T/GAPDH in the cloned skeletal and cardiac implants were 0.22±0.03 and 0.15±0.02 (6 weeks) and 0.09±0.08 and 0.29±0.1 (12 weeks), respectively. In contrast, these expression intensities were significantly lower or absent in constructs generated from genetically unrelated cattle (0.02±0.01 and 0±0.00 at 6 weeks, P<0.005; and 0±0.01 and 0.02±0.1 at 12 weeks, P<0.05; FIGS. 2A, B). The cardiac and skeletal explants also expressed large amounts of desmin and troponin I proteins as determined by western blot analysis (FIGS. 2C, D). Desmin expression intensity was significantly greater in the cloned tissue sections than in the controls (85±1 and 68±4 vs. 30±2 and 16±2 at 6 weeks for the skeletal and cardiac implants, respectively, P<0.001; and 80±3 and 121±24 vs. 53±2 and 52±8 at 12 weeks for the constructs generated from the skeletal and cardiac cells, P<0.05). The expression intensities of troponin I in the cloned and control cardiac muscle explants were 68±4 and 16±2 at 6 weeks (P<0.001), respectively, and 94±7 and 54±12 at 12 weeks (P<0.05).

Western blot analysis of the first-set explants indicated an approximately sixfold greater expression intensity of CD4 in the control than in the cloned constructs at 6 weeks (30±10 and 32±3 for the control skeletal and cardiac implants, respectively, vs. 5±1 and 5±1 for the cloned skeletal and cardiac constructs, P<0.0005), confirming a primary immune response to the control grafts. The mean expression intensities of CD8 were also significantly greater in the control than in the cloned constructs at 6 weeks (26±5 vs. 15±4, P<0.05). Twelve weeks after second-set implantation, mean expression intensities of CD4 and CD8 remained significantly greater in the control than in the cloned constructs (23±4 vs. 12±3, respectively, for CD4, and 54±7 vs. 26±2, respectively, for CD8; P<0.005).

Renal constructs. Renal cells were isolated from a 56-day-old cloned metanephros and passaged until the desired number of cells were obtained. In vitro immunocytochemistry confirmed expression of renal-specific proteins, including synaptopodin (produced by podocytes), aquaporin-1 (AQP1, produced by proximal tubules and the descending limb of the loop of Henle), aquaporin-2 (AQP2, produced by collecting ducts), Tamm-Horsfall protein (produced by the ascending limb of the loop of Henle), and Factor VIII (produced by endothelial cells). Cells expressing synaptopodin and AQP1 or AQP2 exhibited circular and linear patterns in two-dimensional culture, respectively. After expansion, the renal cells produced both erythropoietin and 1,25-dihydroxyvitamin D₃, a key endocrinologic metabolite. The cloned cells produced 2.9±0.03 mIU/ml of erythropoietin (compared with 0.0±0.03 mIU/ml for control fibroblasts (P<0.0005) and 2.9±0.39 mIU/ml for control renal cells) and were responsive to hypoxic stimulation (5.4±1.01 mIU/ml at 1% O₂ vs. 2.9±0.03 mIU/ml at 20% O₂, P<0.02). The concentration of 1,25-dihydroxyvitamin D₃ was 20.2±1.12 pg/ml for the cloned cells, compared with <1 pg/ml for control fibroblasts (P<0.0002) and 18.6±1.72 pg/ml for control renal cells.

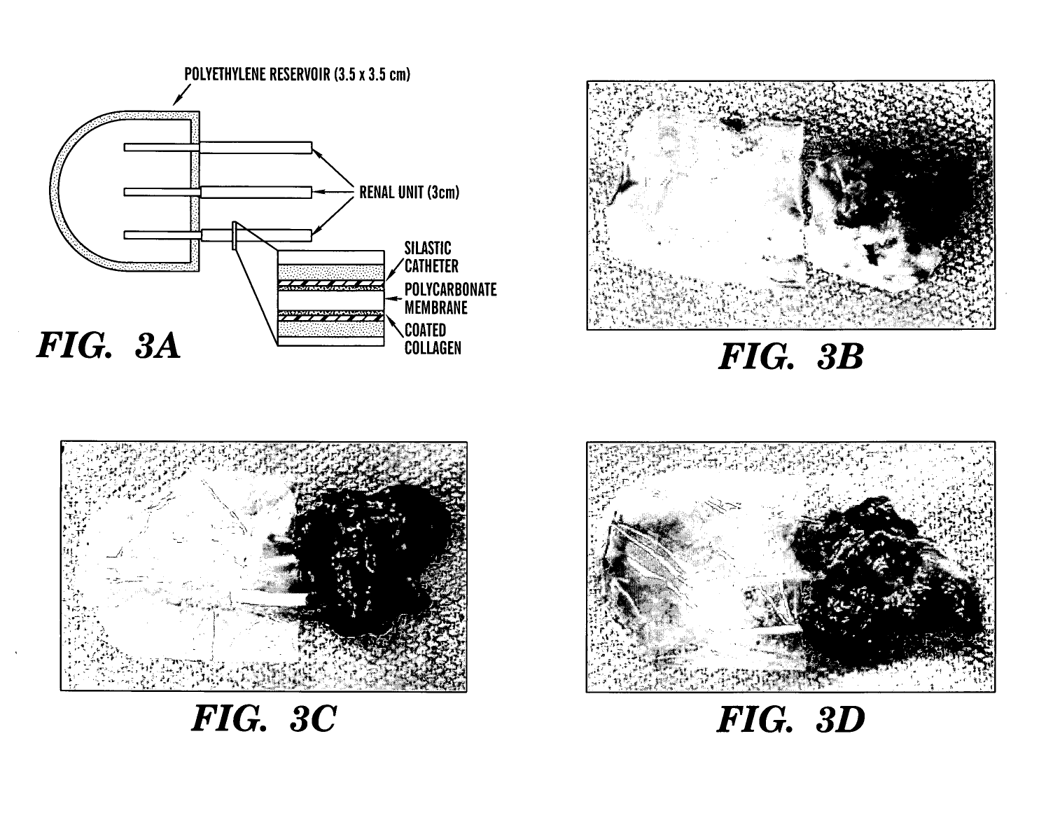

After expansion and characterization, the cloned cells were seeded onto collagen-coated cylindrical polycarbonate membranes. Renal devices with collecting systems were constructed by connecting the ends of three membranes with catheters that terminated in a reservoir (FIG. 3A). A total of 31 units (n=19 with cloned cells, n=6 without cells, and n=6 with cells from an allogeneic control fetus) were transplanted subcutaneously and retrieved 12 weeks after implantation into the nuclear donor animal.

On gross examination, the explanted units appeared intact, and straw-yellow fluid was seen in the reservoirs of the cloned group (FIG. 3D). The volume of fluid produced by the experimental group was sixfold greater than that produced by the control groups (0.60±0.04 ml vs. 0.10±10.01 ml and 0.13±0.04 ml in the allogeneic and unseeded control groups, respectively, P<0.00001). Chemical analysis of the fluid suggested unidirectional secretion and concentration of urea nitrogen (18.3±1.8 mg/dl urea nitrogen in the cloned group vs. 5.6±0.3 mg/dl and 5.0±0.01 mg/dl in the allogeneic and unseeded control groups, respectively, P<0.0005) and creatinine (2.5±0.18 mg/dl creatinine in the cloned group vs. 0.4±0.18 mg/dl and 0.4±0.08 mg/dl in the allogeneic and unseeded control groups, respectively, P<0.0005). Although the ratios of urine to plasma urea and creatinine were not physiologically normal, they were significantly greater than those of the controls, approaching up to 60% of what is considered to be within normal limits (the urine/plasma creatinine ratio was 6:1 in the cloned constructs vs. 10:1 in normal kidneys).

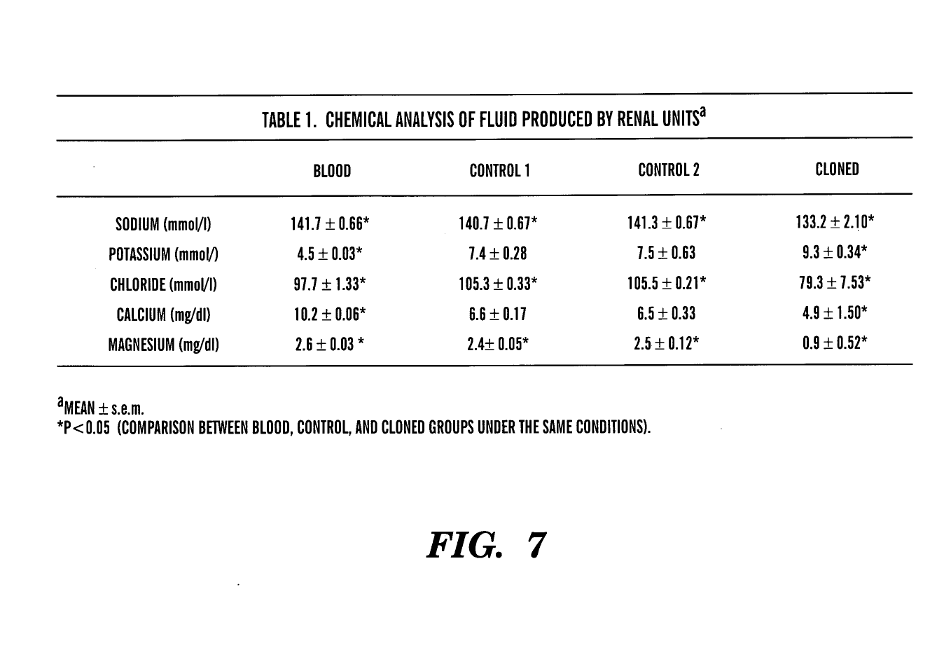

The physiologic function of the implanted units was further demonstrated by analysis of the electrolyte levels, specific gravity, and glucose concentrations of the collected fluid. The electrolyte levels in the fluid of the experimental group were significantly different from those of the plasma and the controls (Table 1, FIG. 7), indicating that the implanted renal cells possessed filtration, reabsorption, and secretory functions. Urine specific gravity is an indicator of kidney function and reflects the action of the tubules and collecting ducts on the glomerular filtrate by giving an estimate of the solute concentration in the urine. The urine specific gravity of cattle is ˜1.025 and normally ranges from 1.020 to 1.040 (as compared with ˜1.010 in normal bovine serum) 36, 37. The specific gravity of the fluid produced by the cloned renal units was 1.027±0.001. The normal range of urine pH for adult herbivores is 7.0-9.0 (ref. 37). The pH of the fluid from the cloned renal units was 8.1±0.20. Glucose is reabsorbed in the proximal tubules and is seldom present in cattle urine. Glucose was undetectable (<10 mg/dl) in the cloned renal fluid (as compared with a blood glucose concentration of 76.6±0.04 mg/dl in the animals in the experimental group). The rate of excretion of minerals in cattle depends on a number of variables, including the mineral concentration in the animals' feed 36. However, the concentrations of magnesium and calcium, which are both reabsorbed in the proximal tubules and the loop of Henle, are normally <2.5 mg/dl and <5 mg/dl in bovine urine, respectively, and were 0.9±0.52 mg/dl and 4.9±1.5 mg/dl in the cloned urinelike fluid, respectively.

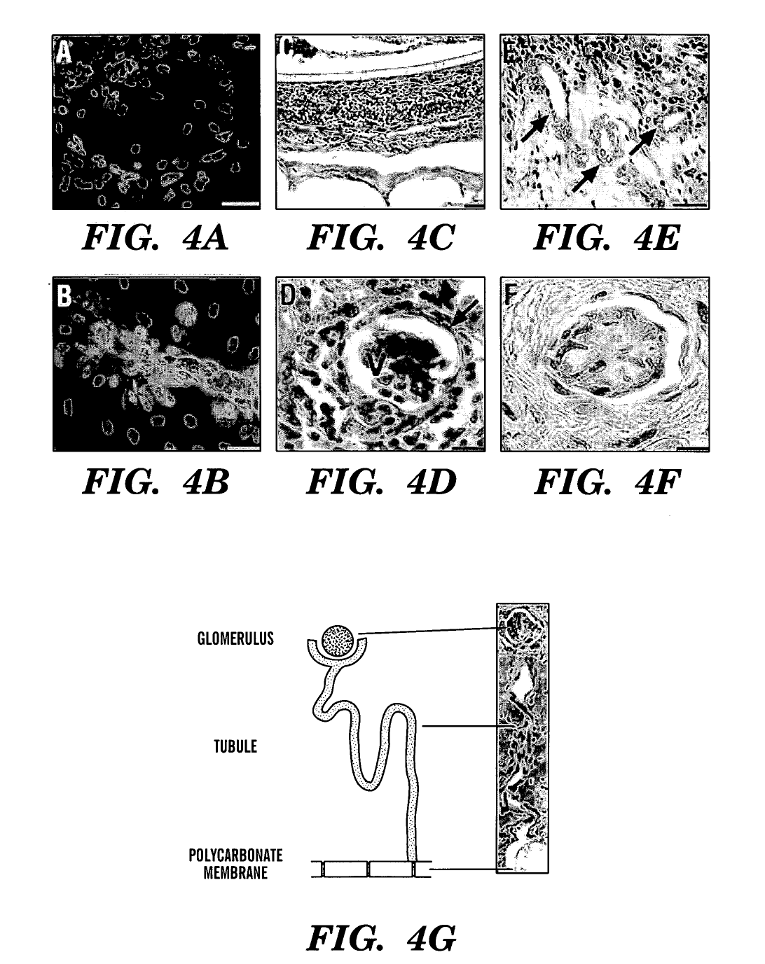

The retrieved implants showed extensive vascularization and had self-assembled into glomeruli and tubule-like structures (FIG. 4). The latter were lined with cuboid epithelial cells with large, spherical, pale-stained nuclei, whereas the glomeruli structures showed a variety of cell types with abundant red blood cells. There was a clear continuity between the mature glomeruli, their tubules, and the polycarbonate membrane (FIG. 4G). The renal tissues were integrally connected in a unidirectional manner to the reservoirs, resulting in the excretion of dilute urine into the collecting systems.

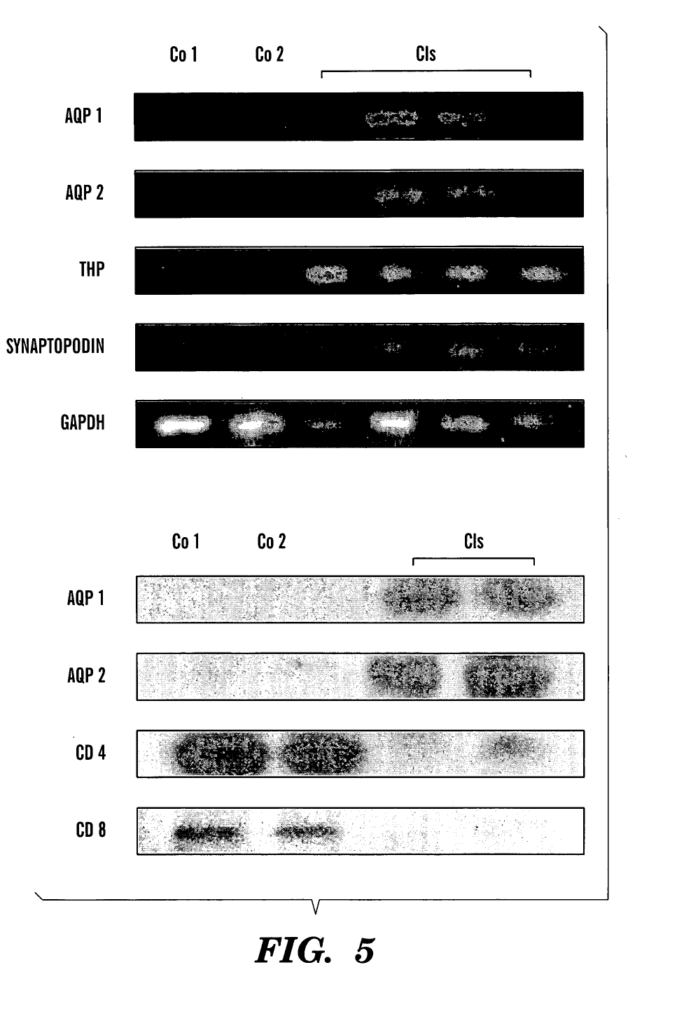

Immunohistochemical analysis confirmed the expression of renal-specific proteins, including AQP1, AQP2, synaptopodin, and Factor VIII (FIG. 4). Antibodies for AQP1, AQP2, and synaptopodin identified tubular, collecting-tubule, and glomerular segments within the constructs, respectively. In contrast, the allogeneic controls displayed a foreign-body reaction with necrosis, consistent with the finding of acute rejection. RT-PCR analysis confirmed the transcription of AQP1, AQP2, synaptopodin, and Tamm-Horsfall genes exclusively in the cloned group (FIG. 5). Cultured and cloned cells also expressed large amounts of AQP1, AQP2, synaptopodin, and Tamm-Horsfall protein as determined by western blot analysis. The expression intensities of CD4 and CD8, markers for inflammation and rejection, were also significantly higher in the control than in the cloned group (FIG. 5).

Mitochondrial DNA (mtDNA) analysis. Previous studies showed that bovine clones harbor the oocyte mtDNA 6-8, 38. As discussed above, differences in mtDNA-encoded proteins expressed by cloned cells could stimulate a T-cell response specific for mtDNA-encoded minor histocompatibility antigens (miHAs) 39 when cloned cells are transplanted back to the original nuclear donor. The most straightforward approach to resolving the question of miHA involvement is the identification of potential antigens by nucleotide sequencing of the mtDNA genomes of the clone and the fibroblast nuclear donor. The contiguous segments of mtDNA that encode 13 mitochondrial proteins and tRNAs were amplified by PCR from total cell DNA in five overlapping segments for both donor-recipient combinations. These amplicons were directly sequenced on one strand with a panel of sequencing primers spaced at 500 bp intervals.

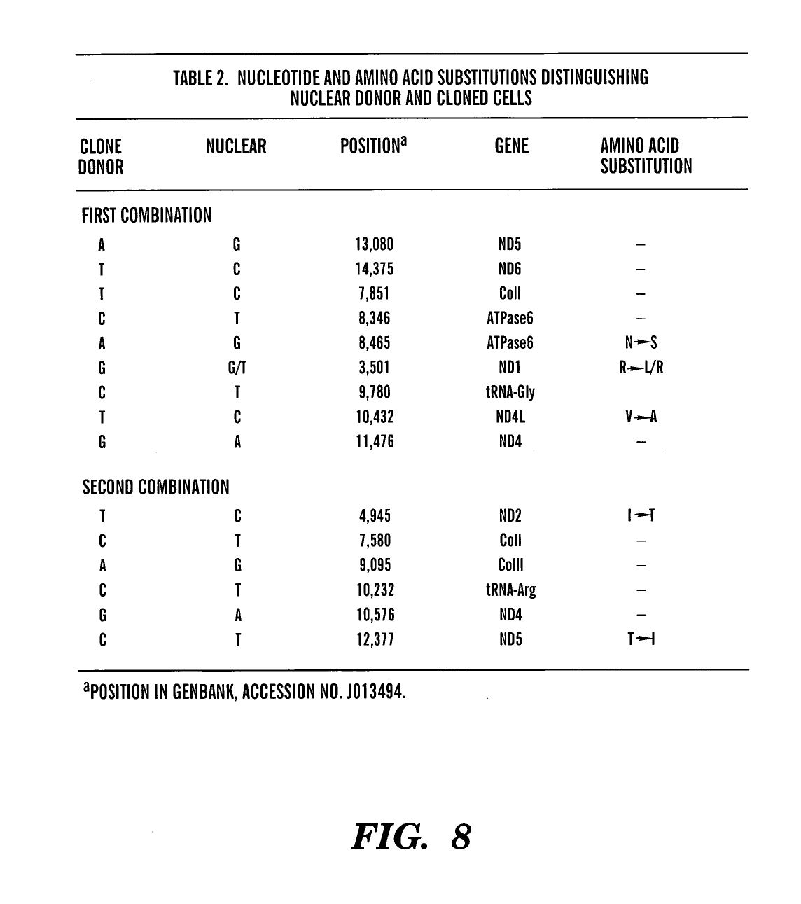

The resulting nucleotide sequences (13,210 bp) revealed nine nucleotide substitutions (Table 2, FIG. 8) for the first donor-recipient combination (cardiac and skeletal constructs). One substitution was in the tRNA-Gly segment, and five substitutions were synonymous. The sixth substitution, in the ND1 gene, was heteroplasmic in the nuclear donor where one of the two alternative nucleotides was shared with the clone. A leucine or arginine would be translated at this position in ND1. The eighth and ninth substitutions resulted in amino acid interchanges of asparagine to serine and valine to alanine in the ATPase6 and ND4L genes, respectively. For the second donor-recipient combination (renal constructs), we obtained 12,785 bp from both the clone and the nuclear donor animal. The resulting sequences revealed six nucleotide substitutions (Table 2, FIG. 8). One substitution was in the tRNA-Arg segment and three substitutions were synonymous. The fifth and sixth substitutions resulted in amino acid interchanges of isoleucine to threonine and threonine to isoleucine in the ND2 and ND5 genes, respectively.

The identification of two amino acid substitutions that distinguish the clone and the nuclear donor confirms that a maximum of only two miHA peptides could be defined for each donor-recipient combination. Given the lack of knowledge about peptide-binding motifs for bovine MHC class I molecules, there is no reliable method to predict the impact of these amino acid substitutions on the ability of mtDNA-encoded peptides either to bind to bovine class I molecules or to activate CD8⁺ cytotoxic T lymphocytes (CTLs).

Despite the potential immunogenicity of the two amino acid substitutions in the first donor-recipient combination, it was clear that the cloned devices functionally survived for the duration of the experiments without significant increases in infiltration of second-set devices by CD4⁺ and CD8⁺ T lymphocytes. Specifically, cloned cardiac and skeletal tissues remained viable for more than three months after second-set transplantation (comparable to in vitro control specimens). Multiple, viable, myosin- and troponin I-containing cells were observed throughout the tissue constructs, consistent with functionally active protein synthesis and expression. This direct assessment of graft function does not provide any evidence to support the activation of a T-cell response to cloned tissue-specific histocompatibility antigens in this donor-recipient combination.

These findings are consistent with those of the second transplant donor-recipient combination. The cloned renal cells derived their nuclear genome from the original fibroblast donor and their mtDNA from the original recipient oocyte. A relatively limited number of mtDNA polymorphisms have been shown to define maternally transmitted miHAs in mice 39. This class of miHAs stimulates both skin allograft rejection in vivo and expansion of CTLs in vitro 39, and might constitute a barrier to successful clinical use of such cloned devices, as has been hypothesized in chronic rejection of MHC-matched human renal transplants 40, 41. We chose to investigate a possible anti-miHA T-cell response to the cloned renal devices through both DTH testing in vivo and Elispot analysis of IFNγ-secreting T cells in vitro. An in vivo assay of anti-miHA immunity was chosen on the basis of the ability of skin allograft rejection to detect a wide range of miHAs in mice with survival times exceeding ten weeks 42 and the relative insensitivity of in vitro assays in detecting miHA incompatibility, highlighted by the requirement for in vivo priming to generate CTLs 43. Using DTH testing in vivo, we did not see an immunological response directed against the cloned cells. Cloned and control allogeneic cells were intradermally injected back into the nuclear donor animal 80 days after the initial transplantation. A positive DTH response was observed after 48 h for the allogeneic control cells but not for the cloned cells (diameter of erythema and induration of about 9×4.5 mm, 12×10 mm, and 11×11 mm vs. 0, 0, and 0 mm, respectively, P<0.02).

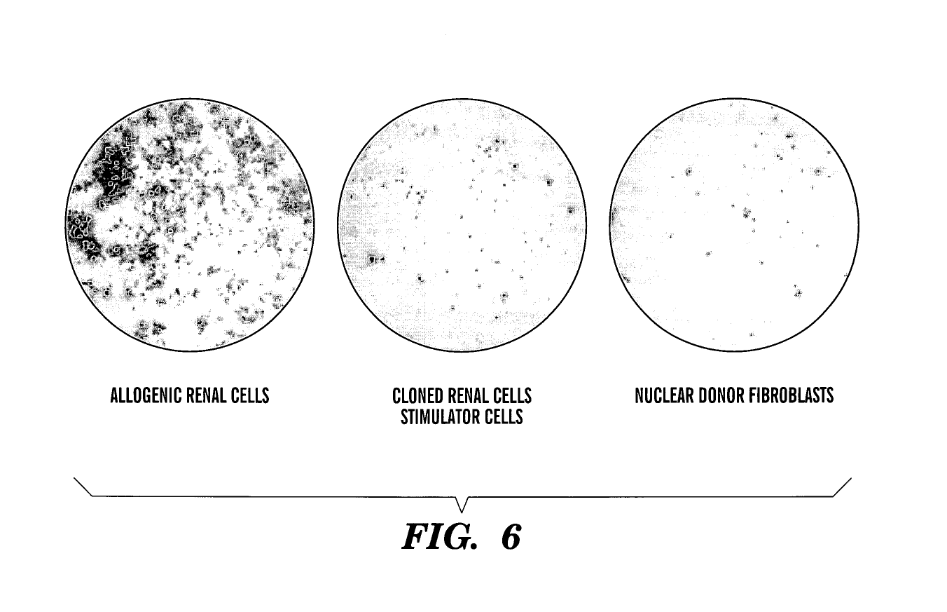

The results of DTH analysis were mirrored by Elispot-derived estimates of the frequencies of T cells that secreted IFNγ after in vitro stimulation. Primary B lymphocytes were harvested from the transplanted recipient one month after retrieval of the devices. These primary B lymphocytes were stimulated in primary mixed-lymphocyte cultures with allogeneic renal cells, cloned renal cells, and nuclear donor fibroblasts. Surviving T cells were restimulated in anti-IFNγ-coated wells with either nuclear donor fibroblasts (autologous control) or the respective stimulators used in the primary mixed-lymphocyte cultures. Elispot analysis revealed a relatively strong T-cell response to allogeneic renal stimulator cells relative to the responses to either cloned renal cells or nuclear donor fibroblasts (FIG. 6). A mean of 342 spots (s.e.m.±36.7) was calculated for allogeneic renal cell-specific T cells. Significantly lower numbers of IFNγ-secreting T cells responded to cloned renal cells and nuclear donor fibroblasts. Nuclear donor fibroblast-stimulated T cells yielded 45 (s.e.m.±1.4) and 55 (s.e.m.±5.7) spots after secondary stimulation with cloned renal and nuclear donor fibroblast stimulators, respectively. Likewise, cloned renal cell-stimulated T cells yielded 61 (s.e.m.±2.8) and 33.5 (s.e.m.±0.7) spots with the same stimulator populations. These results corroborate both the relative CD4 and CD8 expression in western blots (FIG. 5), and the results of in vivo DTH testing, supporting the conclusion that no detectable rejection response specific for cloned renal cells occurred after either primary or secondary challenge.

Conclusions. Our results suggest that cloned cells and tissues with allogeneic mtDNA can be grafted back into the nuclear donor organism without destruction by the immune system, although further studies will be necessary to rule out the possibility of immune rejection with other donor-recipient transplant combinations. It is important to note that bovine ES cells capable of differentiating into specified tissue in vitro have not yet been isolated. It was therefore necessary in the present study to generate an early-stage bovine embryo. This strategy could not be applied in humans, as ethical considerations require that preimplantation embryos not be developed in vitro beyond the blastocyst stage 44-46. However, human and primate ES cells have been successfully differentiated in vitro into derivatives of all three germ layers, including beating cardiac muscle cells, smooth muscle, and insulin-producing cells, among others 47, 52.

Although functional tissues can be engineered using adult native cells 53, 54, the ability to bioengineer primordial stem cells into more complex functional structures such as kidneys would overcome the two major problems in transplantation medicine: immune rejection and organ shortage. It is clear that a staged developmental strategy will be required to achieve this ultimate goal. The results presented here suggest that nuclear transplantation may overcome the hurdle of immune incompatibility.

Experimental Protocol

Adult bovine cell line derivation. Dermal fibroblasts were isolated from adult Holstein steers by ear notch. Tissue samples were minced and cultured in DMEM (Gibco, Grand Island, N.Y.) supplemented with 15% FCS (HyClone, Logan, Utah), L-glutamine (2 mM), nonessential amino acids (100 μM), β-mercaptoethanol (154 μM), and antibiotics at 38° C. in a humidified atmosphere of 5% CO₂ and 95% air. The tissue explants were maintained in culture and a fibroblast cell monolayer established. The cell strain was maintained in culture, passaged, cryopreserved in 10% dimethyl sulfoxide, and stored in liquid nitrogen before nuclear transfer. Experimental protocols followed guidelines approved by the Children's Hospital (Boston, Mass.) and Advanced Cell Technology (Worcester, Mass.) Institution Animal Care and Use Committees.

Nuclear transfer and embryo culture. Bovine oocytes were obtained from abattoir-derived ovaries as described elsewhere 38. Oocytes were mechanically enucleated at 18-22 h post maturation, and complete enucleation of the metaphase plate was confirmed with bisbenzimide (Hoechst 33342; Sigma, St. Louis, Mo.) dye under fluorescence microscopy. A suspension of actively dividing cells was prepared immediately before nuclear transfer. Single donor cells were selected and transferred into the perivitelline space of the enucleated oocytes. Fusion of the cell-oocyte complexes was accomplished by applying a single pulse of 2.4 kV/cm for 15 μs. Nuclear transfer embryos were activated as described elsewhere by Presicce et al. 55 with slight modifications. Briefly, reconstructed embryos were exposed to 5 μM ionomycin (CalBiochem, La Jolla, Calif.) in Tyrode lactate-HEPES for 5 min at room temperature followed by a 6 h incubation with 5 μg/ml cytochalasin B (Sigma) and 10 μg/ml cycloheximide (Sigma) in astroglial cell-culture medium. The resulting blastocysts were nonsurgically transferred into progestin-synchronized recipients.

Cell culture and seeding. Cardiac and skeletal tissue from five- to six-week-old cloned and natural fetuses were retrieved. The cells were isolated by the explant technique and cultured using DMEM as above. Both muscle cell types were expanded separately until desired numbers of cells were obtained. The cells were trypsinized, washed, and seeded in 1×2 cm PGA polymer scaffolds with 5×10⁷ cells. Vials of frozen donor cells were thawed and passaged before seeding the second-set scaffolds. Renal cells were derived from seven- to eight-week-old cloned and natural fetuses. Metanephros were surgically dissected under a microscope, and cells were isolated by enzymatic digestion using 0.1% (wt/vol) collagenase/dispase (Roche, Indianapolis, Ind.) and cultured using DMEM supplemented as above. Cells were passed by 1:3 or 1:4 every three to four days, and expanded until desired cell numbers (˜6×10⁸) were obtained. The cells were seeded in coated collagen with 2×10⁷ cells/cm² density. Vials of frozen donor cells were thawed and passaged for DTH testing and for use in the in vitro proliferative assays.

Polymers and renal devices. Unwoven sheets of polyglycolic acid polymers (1 cm×2 cm×3 mm) were used as cell delivery vehicles (Albany International, Mansfield, Mass.). The polymer meshes were composed of fibers 15 μm in diameter with an interfiber distance of 0-200 μm with 95% porosity. The scaffold was designed to degrade by hydrolysis in 8-12 weeks. Renal devices with collecting systems were constructed by connecting the ends of three cylindrical polycarbonate membranes (3 cm long, 10 μm thick, 2 μm pore size, 1.4 mm internal diameter; Nucleopore Filtration Products, Cambridge, Mass.) with 16 G Silastic catheters that terminated in a 2 ml reservoir made from polyethylene sealed along the edge by the application of pressure and heat. The distal end of the cylindrical membranes was also sealed, and the membranes coated with type 1 collagen (0.2 cm thickness) extracted from rat-tail collagen.

Implantation and analysis of fluid. The cell-polymer constructs were implanted into the flank subcutaneous tissue of the same steer from which the cells were cloned. Fourteen constructs (eight first-set and six second-set) for each cell type were implanted. Control group constructs, with cells isolated from an allogeneic fetus, were implanted on the contralateral side. The implanted constructs were retrieved at 6 weeks (first set) and 12 weeks (second set) after implantation. The renal units were also derived from a single fetus. Thirty-one units (n=19 with cloned cells, n=6 without cells, and n=6 with cells isolated from an allogeneic, age-matched control fetus) were transplanted subcutaneously and retrieved 12 weeks after implantation. The solute concentrations of urea nitrogen, creatinine, and electrolytes were measured in the accumulated fluid in the explanted renal reservoirs using standard techniques.

DTH testing. Cloned, allogeneic, and autologous cells were intradermally injected into the nuclear donor animal (1×10⁶ cells in 0.1 ml in triplicate). Three sites were chosen for softest skin: the left and right side of the tail, and just below the anus. After each site was shaved and prepared, the cells were injected in a row about 2 cm apart. The area of erythema and induration was measured (blinded) after 24-72 h, with 48 h being considered the optimal time to detect a DTH response.

Elispot analysis. Bovine recipient peripheral blood lymphocytes (PBLs) were isolated from whole blood and cultured for six days with irradiated allogeneic renal cells, cloned renal cells, and nuclear donor fibroblasts at 37° C. in RPMI medium plus 10% FCS and human interleukin-2 (20 units/ml) (Chiron, Emeryville, Calif.). On day 6, the stimulated PBLs were harvested and plated at 25,000 cells/well in duplicate wells of a 96-well Multiscreen plate, which had been coated overnight with mouse anti-bovine IFNγ (10 μg/ml) (Biosource, Camarillo, Calif.). A total of 50,000 cells matched to the primary culture stimulators were added to the respective wells. The plate was incubated for 24 h at 37° C. and washed 3× with 0.5% Tween-20 and 4× in distilled water. Biotinylated mouse anti-bovine IFNγ (5 μg/ml) (Biosource) was added, and the plate was incubated for 2 h at 37° C. The plate was washed as above and alkaline phosphatase-conjugated anti-biotin (1:1000 dilution; Vector, Burlingame, Calif.) was added and incubated for 1 h at room temperature. The plate was washed and 100 μl of 5-bromo-4-chloro-3-indolyl phosphate/nitro blue tetrazolium (BCIP/NBT) (Sigma) was added for development of spots. After development, BCIP/NBT was washed out of the wells with distilled water. The wells were photographed and analyzed with Immunospot software (Cellular Technologies, Cleveland, Ohio).

Histological and immunohistochemical analyses. Sections (5 μm) of 10% (wt/vol) buffered formalin-fixed paraffin-embedded tissue were cut and stained with hematoxylin and eosin (H&E). Immunohistochemical analyses were done with specific antibodies to identify the cell types in retrieved tissues with cryostat and paraffin sections. Monoclonal sarcomeric tropomyosin (Sigma) and troponin I (Chemicon, Temecula, Calif.) antibodies were used to detect skeletal and cardiac fibers, respectively. Monoclonal synaptopodin (Research Diagnostics, Flanders, N.J.), polyclonal AQP1 and AQP2, and polyclonal Tamm-Horsfall protein (Biomedical Technologies, Stoughton, Mass.) were used to detect glomerular and tubular tissue, respectively. Monoclonal CD4 and CD8 (Serotec, Raleigh, N.C.) antibodies were used to identify T cells for immune rejection. Specimens were routinely processed for immunostaining. Pretreatment for high-temperature antigen unmasking pretreatment with 0.1% trypsin was conducted using a commercially available kit according to the manufacturer's recommendations (T-8128; Sigma). Antigen-specific primary antibodies were applied to the deparaffinized and hydrated tissue sections. Negative controls were treated with nonimmune serum instead of the primary antibody. Positive controls consisted of normal tissue. After washing with PBS, the tissue sections were incubated with a biotinylated secondary antibody and washed again. A peroxidase reagent (diaminobenzidine) was added. Upon substrate addition, the sites of antibody deposition were visualized by a brown precipitate. Counterstaining was performed with Gill's hematoxylin. To determine the degree of immunoreaction, the immune cells were counted under five high-power fields per section (HPF, ×200) using computerized histomorphometrics (BioImaging Analyses Software, NIH Image 6.2, NIH, Rockville, Md.).

Erythropoietin and 1,25-dihydroxyvitamin D₃ assays. Cloned renal cells, allogeneic renal cells, and cloned fibroblasts were grown to confluence in 60 mm culture dishes (in quadruplicate) at 20% O₂, 5% CO₂. After washing 3×, the cells were incubated in either serum-free medium for 24 h (erythropoietin) or serum-free medium with 1,25-hydroxyvitamin D₃ (1 ng/ml) for 12 h. Erythropoietin production in the supernatants was measured by the double-antibody sandwich enzyme-linked immunosorbent assay (ELISA) using a Quantikine IVD Erythropoietin ELISA kit (R&D Systems, Minneapolis, Minn.). Erythropoietin production was also measured in the supernatant of cells that were incubated in a hypoxic chamber (1% O₂, 5% CO₂) for 4 h. Production of 1,25-dihydroxyvitamin D₃ in the supernatants was measured by radioimmunoassay using a ¹²⁵I RIA kit (DiaSorin, Stillwater, Minn.).

Mitochondrial DNA analyses. Mitochondrial DNA products ranging in size from 3 kb to 3.8 kb were amplified by PCR using Advantage-GC Genomic Polymerase (Clontech, Palo Alto, Calif.) and total genomic DNA templates from the clone and nuclear donor. The regions of the mitochondria that were amplified included all of the protein-coding sequences and the intervening tRNAs. PCR products were electrophoresed in 1% (wt/vol) SeaPlaque GTG agarose (Rockland, Me.), extracted from the gels with the use of QIAquick Gel Extraction Kits (Qiagen, Valencia, Calif.), and sequenced by the Molecular Biology Core Facility (Mayo Clinic, Rochester, Minn.) with a series of primers located at ˜500-base intervals.

RNA isolation and cDNA synthesis. Freshly retrieved tissue implants were harvested and frozen immediately in liquid nitrogen. The tissue was homogenized in RNAzol reagent (Tel-Test, Friendswood, Tex.) at 4° C. using a tissue homogenizer. RNA was isolated according to the manufacturer's protocol (Tel-Test). Complementary DNA was synthesized from 2 g RNA using the Superscript II reverse transcriptase (Gibco) and random hexamers as primers.

PCR. For PCR amplification, 1 ml of cDNA with 1 unit Taq DNA polymerase (Roche), 200 mM dNTP, and 10 pM of each primer were used in a final volume of 30 ml. Myosin for skeletal muscle tissue was amplified from cDNA with primers 5′-TGAATTCAAGGAGGCGTTTCT-3′ (SEQ ID NO: 1) and 5′-CAGGGCTTCCACTTCTTCTTC-3′ (SEQ ID NO: 2). Troponin T for cardiac tissue was done with primers 5′-AAGCGCATGGAGAAGGACCTC-3′ (SEQ ID NO: 3) and 5′-GGATGTAGCCGCCGAAGTG-3′ (SEQ ID NO: 4). Synaptopodin for glomerulus was amplified from cDNA with primers 5′-GGTGGCCAGTGAGGAGGAA-3′ (SEQ ID NO: 5) and 5′-TGCTCGCCCAGACATCTCTT-3′ (SEQ ID NO: 6). Podocalyxin for glomerulus was done with primers 5′-CTCTCGGCGCTGCTGCTACT-3′ (SEQ ID NO: 7) and 5′-CGCTGCTGGTCCTTCCTCTG-3′ (SEQ ID NO: 8). AQP1 for tubule was done with primers 5′-CAGCATGGCCAGCGACGAGTTCAAGA-3′ (SEQ ID NO: 9) and 5′-TGTCGTCGGCATCCAGGTCATAC-3′ (SEQ ID NO: 10); AQP2 for tubule was done with primers 5′-GCAGCATGTGGGARCTNM-3′ (SEQ ID NO: 11) and 5′-CTYACIGCRTTIACNGCNAGRTC-3′ (SEQ ID NO: 12). Tamm-Horsfall protein for tubule was done with primers 5′-AACTGCTCCGCCACCAA-3′ (SEQ ID NO: 13) and 5′CTCACAGTGCCTTCCGTCTC-3′ (SEQ ID NO: 14). PCR products were visualized with agarose gel electrophoresis and ethidium bromide staining.

Western blot analysis. Tissue was homogenized in lysis buffer using a tissue homogenizer. After measuring protein concentration (Bio-Rad), equal protein amounts were loaded on 10% SDS-PAGE. Proteins were blotted onto polyvinylidene fluoride membranes, which were incubated with primary antibodies for 1 h at room temperature. Desmin (Santa Cruz Biotech, Santa Cruz, Calif.) antibodies were used to detect skeletal tissue; desmin and troponin I (Santa Cruz Biotech) antibodies were used to detect cardiac tissue; and synaptopodin, AQP1, AQP2, and Tamm-Horsfall protein (Research Diagnostics, Flanders, N.J.) were used to detect glomerular and tubular tissue, respectively. Monoclonal CD4 and CD8 antibodies were used as markers for inflammation and rejection. Subsequently, membranes were incubated with secondary antibodies for 30 min. The signal was visualized using the ECL system (NEN, Boston, Mass.).

Statistical analysis. Data are presented as mean±s.e.m. and compared using the two-tailed Student's t-test. Differences were considered significant at P<0.05.

### REFERENCES

The references cited herein and throughout the specification are herein incorporated by reference in their entirety.
1. Lanza, R. P. et al. The ethical reasons for stem cell research. Science 293, 1299 (2001)
2. Atala, A. & Lanza, R. P. Methods of Tissue Engineering (Academic Press, San Diego, Calif., 2001).
3. Atala, A. & Mooney, D. Synthetic Biodegradable Polymer Scaffolds (Birkhäuser, Boston, Mass., 1997).
4. Machluf, M. & Atala, A. Emerging concepts for tissue and organ transplantation. Graft 1, 31-37 (1998).
5. Lanza, R. P., Cibelli, J. B. & West, M. D. Prospects for the use of nuclear transfer in human transplantation. Nat. Biotechnol. 17, 1171-1174 (1999).
6. Evans, M. J. et al. Mitochondrial DNA genotypes in nuclear transfer-derived cloned sheep. Nat. Genet. 23, 90-93 (1999).
7. Hiendleder, S., Schmutz, S. M., Erhardt, G., Green, R. D. & Plante, Y. Transmitochondrial differences and varying levels of heteroplasmy in nuclear transfer cloned cattle. Mol. Reprod. Dev. 54, 24-31 (1999).
8. Steinborn, R. et al. Mitochondrial DNA heteroplasmy in cloned cattle produced by fetal and adult cell cloning. Nat. Genet. 25, 255-257 (2000).
9. Vyas, J. M. et al. Biochemical specificity of H-2M3a: stereospecificity and space-filling requirement at position 1 maintains N-formyl peptide binding. J. Immunol. 149, 3605-3611 (1992).
10. Morse, M. et al. The COI mitochondrial gene encodes a minor histocompatibility antigen presented by H2-M3. J. Immunol. 156, 3301-3307 (1996).
11. Loveland, B., Wang, C. R., Yonekawa, H., Hermel, E. & Lindahl, K. F. Maternally transmitted histocompatibility antigens of mice: a hydrophobic peptide of a mitochondrial encoded protein. Cell 60, 971-980 (1990).
12. Davies, J. D. et al. Generation of T cells with lytic specificity for atypical antigens. I. A mitochondrial antigen in the rat. J. Exp. Med 173, 823-832 (1991).
13. Lysaght, M. J. Maintenance dialysis population dynamics: current trends and long-term implications. J. Am. Soc. Nephrol. 13, S37-S40 (2002).
14. Amiel, G. E. & Atala, A. Current and future modalities for functional renal replacement. Urol. Clin. 26, 235-246 (1999).
15. Humes, H. D., Buffington, D. A., MacKay, S. M., Funke, A. J. & Weitzel, W. F. Replacement of renal function in uremic animals with a tissue-engineered kidney. Nat. Biotechnol. 17, 451-455 (1999).
16. Cieslinski, D. A. & Humes, H. D. Tissue engineering of a bioartificial kidney. Biotechnol. Bioeng. 43, 781-791 (1994).
17. MacKay, S. M., Kunke, A. J., Buffington, D. A. & Humes, H. D. Tissue engineering of a bioartificial renal tubule. ASAIO J. 44, 179-183 (1998).
18. Aebischer, P., Ip, T. K., Panol, G. & Galletti, P. M. The bioartificial kidney: progress towards an ultrafiltration device with renal epithelial cells processing. Life Support Syst. 5, 159-168 (1987).
19. Ip, T., Aebischer, P. & Galletti, P. M. Cellular control of membrane permeability. Implications for a bioartificial renal tubule. ASAIO Trans. 34, 351-355 (1988).
20. Humes, H. D. Renal replacement devices. in Principles of Tissue Engineering; Edn. 2 (eds Lanza, R. P., Langer, R. & Vacanti, J.) 645-653 (Academic Press, San Diego, 2000).
21. Amiel, A., Yoo, J. & Atala, A. Renal therapy using tissue engineered constructs and gene delivery. World J. Urol. 18, 71-79 (2000).
22. Lanza, R. P., Hayes, J. L. & Chick, W. L. Encapsulated cell technology. Nat. Biotechnol. 14, 1107-1111 (1996).
23. Kuhtreiber, W. M., Lanza, R. P. & Chick, W. L. (eds). Cell Encapsulation Technology and Therapeutics (Birkhauser, Boston, 1998).
24. Lanza, R. P & Chick, W. L. (eds). Immunoisolation of Pancreatic Islets (R. G. Landes, Austin, Tex., 1994).
25. Joki, T. et al. Continuous release of endostatin from microencapsulated engineered cells for tumor therapy. Nat. Biotechnol. 19, 35-39 (2001).
26. Qiao, J., Sakurai, H. & Nigam, S. K. Branching morphogenesis independent of mesenchymal-epithelial contact in the developing kidney. Proc. Natl. Acad. Sci. USA 96, 7330-7335 (1999).
27. Humes, H. D., Krauss, J. C., Cieslinski, D. A. & Funke, A. J. Tubulogenesis from isolated single cells of adult mammalian kidney: clonal analysis with a recombinant retrovirus. Am. J. Physiol. 271, F42-F49 (1996).
28. Lanza, R. P, Langer, R. & Vacanti, J. Principles of Tissue Engineering (Academic Press, San Diego, Calif., 2000).
29. Atala, A. Future perspectives in reconstructive surgery using tissue engineering. Urol. Clin. 26, 157-166 (1999).
30. Santavirta, S. et al. Immune response to polyglycolic acid implants. J. Bone Joint Surg. Br. 72, 597-600 (1990).
31. Paivarinta, U. et al. Intraosseous cellular response to biodegradable fracture fixation screws made of polyglycolide or polylactide. Arch. Orthop. Trauma Surg. 112, 71-74 (1993).
32. Bostman, O. M. & Pihlajamaki, H. K. Adverse tissue reactions to bioabsorbable fixation devices. Clin. Orthop. 371, 216-227 (2000).
33. Ruuskanen, M. et al. Evaluation of self-reinforced polyglycolide membrane implanted in the subcutis of rabbits. Ann. Chir. Gynaecol. 88 308-312 (1999).
34. Weiler, A., Helling, H. J., Kirch, U., Zirbes, T. K. & Rehm, K. E. Foreign-body fracture fixation: experimental study in sheep. J. Bone Joint Surg. Br. 78, 369-376 (1996).
35. Pariente, J. L., Kim, B. S. & Atala, A. In vitro compatibility assessment of naturally-derived and synthetic biomaterials using normal human urothelial cells. J. Biomed. Mat. Res. 55, 33-39 (2001).
36. Rosenberger, G. Clinical Examination of Cattle (Verlag Paul Parey, Berlin, 1979), pp. 275-281.
37. Smith, B. P. Large Animal Internal Medicine: Diseases of Horses, Cattle, Sheep and Goats, Edn. 2 pp. 467-469 (Mosby, St. Louis, 1996).
38. Lanza, R. P. et al. Cloning of an endangered species (Bos gaurus) using interspecies nuclear transfer. Cloning 2, 79-90 (2000). |Article|
39. Fischer Lindahl, K., Hermel, E., Loveland, B. E. & Wang, C. R. Maternally transmitted antigen of mice. Ann. Rev. Immunol. 9, 351-372 (1991). |Article|
40. Hadley, G. A., Linders, B. & Mohanakumar, T. Immunogenicity of MHC class I alloantigens expressed on parenchymal cells in the human kidney. Transplantation 54, 537-542 (1992).
41. Yard, B. A. et al. Analysis of T cell lines from rejecting renal allografts. Kidney Int. 43, S133-SI38 (1993).
42. Bailey, D. W. Genetics of histocompatibility in mice. I. New loci and congenic lines. Immunogenetics 2, 249-256 (1975).
43. Mohanakumar, T. The Role of MHC and Non-MHC Antigens in Allograft Immunity pp. 1-115 (R. G. Landes Company, Austin, Tex., 1994).
44. Lanza, R. P., Cibelli, J. B. & West, M. D. Human therapeutic cloning. Nat. Med. 5, 975-977 (1999).
45. Cibelli, J. B. et al. Somatic cell nuclear transfer in humans: pronuclear and early embryonic development. e-biomed: J. Regen. Med. 2, 25-31 (2001).
46. Lanza, R. P. et al. The ethical validity of using nuclear transfer in human transplantation. JAMA 284, 3175-3179 (2000).
47. Itskovitz-Eldor, J. et al. Differentiation of human embryonic stem cells into embryoid bodies comprising the three embryonic germ layers. Mol. Med. 5, 88-95 (2000).
48. Schuldiner, M., Yanuka, O., Itskovitz-Eldor, J., Melton, D. A. & Benvenisty, N. Effects of eight growth factors on the differentiation of cells derived from human embryonic stem cells. Proc. Natl. Acad. Sci USA 97, 11307-11312 (2000).
49. Kaufman, D. S. et al. Directed differentiation of human embryonic stem cells into hematopoietic colony forming cells. Blood 94 (Suppl. 1, part 1 of 2), 34a (1999).
50. Reubinoff, B. E. et al. Neural progenitors from human embryonic stem cells. Nat. Biotechnol. 19, 1134-1140 (2001).
51. Reubinoff, B. E., Pera, M. F., Fong, C. Y., Trounson, A. & Bongso, A. Embryonic stem cell lines from human blastocysts: somatic differentiation in vitro. Nat. Biotechnol. 18, 399-404 (2000).
52. Cibelli, J. B. et al. Parthenogenetic stem cells in nonhuman primates. Science 295, 819 (2002).
53. Oberpenning, F. O., Meng, J., Yoo, J. & Atala, A. De novo reconstitution of a functional urinary bladder by tissue engineering. Nat. Biotechnol. 17, 149-155 (1999).
54. Kaushal, S. et al. Circulating endothelial cells for tissue engineering of small diameter vessels. Nat. Med. 7, 1035-1040 (2001).
55. Presicce, G. A. & Yang, X. Parthenogenetic development of bovine oocytes matured in vitro for 24 hr and activated by ethanol and cycloheximide. Mol. Reprod. Dev. 38, 380-385 (1994).

We claim:

1. A tissue-engineered construct for supplementing or replacing a damaged organ, the construct having the following characteristics:
(a) differentiated cells on a three-dimensional biocompatible scaffold, wherein the differentiated cells originated from transferred pluripotent cells; and
(b) at least one physiological function of the organ.

2. The tissue-engineered construct of claim 1, wherein the damaged organ is select from the group consisting of kidney, heart, liver, spleen, pancreas, bladder, ureter and urethra.

3. The tissue-engineered construct of claim 1, wherein the scaffold comprises a polymer, hydrogel or decellularized tissue.

4. The tissue-engineered construct of claim 1, wherein the pluripotent cells are differentiated to result in a desired cell type prior to contact with the scaffold.

5. The tissue-engineered construct of claim 1, wherein the pluripotent cells are human stem cells.

6. The tissue-engineered construct of claim 5, wherein the human stem cells are selected from the group consisting of pluripotent hematopoietic stem cells, embryonic stem cells and adult somatic stem cells.

7. The tissue-engineered construct of claim 1, wherein the pluripotent cells are obtained from tissues selected from the group consisting of bone marrow, muscle, adipose tissue, liver, heart, lung and nervous system.

8. The tissue-engineered construct of claim 7, wherein the tissues are selected from the group consisting of adult, embryonic or fetal tissues.

9. The tissue-engineered construct of claim 1, wherein the construct is selected to supplement the activity of a kidney and the physiological function of the organ is excretion of metabolic waste.

10. The tissue-engineered construct of claim 9, wherein the scaffold comprises a porous membrane structure having an external surface defining an enclosed internal space having at least one effluent channel extending from the construct.

11. A method of producing a tissue-engineered construct for supplementing or replacing a damaged organ comprising:
(a) contacting pluripotent cells with a three-dimensional biocompatible scaffold such that the cells attach to the scaffold;
(b) placing the pluripotent cells under conditions to result in differentiation to a desired cell type; and
(c) culturing the cells attached to the scaffold to produce a tissue layer having at least one physiological function of the organ, thereby producing a tissue-engineered construct.

12. The method of claim 11, wherein the differentiated cells originated from transferred pluripotent cells and said pluripotent cells were placed under conditions that caused differentiation.

13. The method of claim 11, wherein the damaged organ is select from the group consisting of kidney, heart, liver, spleen, pancreas, bladder, ureter and urethra.

14. The method of claim 11, wherein the scaffold comprises a polymer, hydrogel or decellularized tissue.

15. The method of claim 11, wherein the pluripotent cells are differentiated to result in a desired cell type prior to contact with the scaffold.

16. The method of claim 11, wherein the pluripotent cells are human stem cells.

17. The method of claim 16, wherein the human stem cells are selected from the group consisting of pluripotent hematopoietic stem cells, embryonic stem cells and adult somatic stem cells.

18. The method of claim 11, wherein the pluripotent cells are obtained from tissues selected from the group consisting of bone marrow, muscle, adipose tissue, liver, heart, lung and nervous system.

19. The method claim 18, wherein the tissues are selected from the group consisting of adult, embryonic or fetal tissues.

20. The method of claim 11, wherein the construct is selected to supplement the activity of a kidney and the physiological function of the organ is excretion of metabolic waste.

21. The method of claim 11, wherein the scaffold comprises a porous membrane structure having an external surface defining an enclosed internal space having at least one effluent channel extending from the construct.
